(19) United States
(12) Patent Application Publication
(10) Pub. No.: US 2005/0215942 A1
(43) Pub. Date: Sep. 29, 2005

(54) Small vessel ultrasound catheter

(76) Inventors: Tim Abrahamson, Seattle, WA (US); Thomas J. Hiblar, Everett, WA (US); Francisco S. Villar, Union City, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/047,464

(22) Filed: Jan. 31, 2005

Related U.S. Application Data
(60) Provisional application No. 60/539,954, filed on Jan. 29, 2004.
(60) Provisional application No. 60/570,969, filed on May 14, 2004.

(51) Int. Cl. A61B 17/20

(52) U.S. Cl. 604/22

(57) ABSTRACT

An ultrasound catheter adapted for accessing small vessels in the distal anatomy is disclosed. The ultrasound catheter comprises an elongate tubular body formed with a delivery lumen. The flexibility and dimensions of the tubular body allow access to the distal anatomy by advancement over the guidewire. An ultrasound radiating member is provided along the distal end portion of the tubular body for emitting ultrasound energy at a treatment site. A drug solution may also be delivered through the delivery lumen and out an exit port to the treatment site.

### PRIORITY APPLICATIONS

This application claims the benefit of U.S. Provisional Application 60/539,954 (filed 29 Jan. 2004; Attorney Docket EKOS.168PR) and U.S. Provisional Application 60/570,969 (filed 14 May 2004; Attorney Docket EKOS.168PR3). All of these priority applications are hereby incorporated by reference herein in their entirety.

### FIELD OF THE INVENTION

The present invention in certain embodiments relates generally to an ultrasound catheter, and specifically to an ultrasound catheter having a variable flexibility along the catheter body.

### BACKGROUND OF THE INVENTION

Ultrasonic energy can be used to enhance the delivery and effect of various therapeutic compounds. Often, an ultrasound catheter delivers ultrasonic energy and/or a therapeutic compound to a treatment site within a patient's vasculature. Such an ultrasound catheter typically comprises an elongate member configured for advancement through a patient's vasculature. An ultrasound assembly is mounted along the distal end portion of the elongate member and is adapted for emitting ultrasonic energy. The ultrasound catheter can include a delivery lumen for delivering the therapeutic compound to the treatment site. In this manner, ultrasonic energy can be delivered to the treatment site to enhance the effect and/or delivery of the therapeutic compound.

For example, in one application, ultrasound catheters have been successfully used to treat human blood vessels that have become occluded by plaque, thrombi, emboli or other substances that reduce the blood carrying capacity of the vessel. See, for example, U.S. Pat. No. 6,001,069. To remove the blockage, the ultrasound catheter is advanced through the patient's vasculature to deliver solutions containing dissolution compounds directly to the blockage site. To enhance the therapeutic effects of the dissolution compound, ultrasonic energy is emitted into the dissolution compound and/or the surrounding tissue. In other applications, ultrasound catheters can be used for other purposes, such as delivering and activating light activated drugs with ultrasonic energy. See, for example, U.S. Pat. No. 6,176,842.

### SUMMARY OF THE INVENTION

Generally, conventional ultrasound catheters are not well adapted for effective use within small blood vessels, such as blood vessels located in the distal anatomy or in the brain. This is often the result of several factors. For example, the distal end portion of the catheter, on which the ultrasound assembly is usually located, is relatively rigid and therefore often lacks sufficient flexibility for navigation through difficult regions of the distal anatomy. In particular, this distal rigidity is generally attributable to the ultrasound radiating member mounted in the distal region of the catheter. Even in an ultrasound assembly having a single ultrasound radiating member, the increased rigidity along the length of the ultrasound radiating member can adversely effect catheter maneuverability. Similarly, the minimum diameter vessel through which an ultrasound catheter can be passed depends, at least in part, on the outer diameter of the ultrasound radiating member. Furthermore, various wires must extend through the catheter to provide power to the ultrasound radiating member. In addition, the ultrasound catheter is typically provided with an inner member further increasing the stiffness of the catheter.

Furthermore, it is difficult to manufacture an ultrasound catheter having a sufficiently small diameter for use in small vessels while still providing the catheter with adequate “pushability” and “torqueability” Likewise, it is difficult to manufacture an ultrasound radiating member having sufficiently small dimensions for use in small vessels while still being capable of generating sufficient quantities of acoustic energy to enhance lysis at the treatment site. Still further, the distal tip of an ultrasound catheter can easily damage the fragile walls of small vessels in the patient's vasculature.

Accordingly, certain embodiments of an improved ultrasound catheter disclosed herein are capable of safely and effectively navigating small blood vessels, such as the main and subsequent branches of the middle cerebral artery. Such an improved catheter is also capable of delivering adequate ultrasonic energy to achieve a desired therapeutic effect. The embodiments described herein illustrate various features of such an improved ultrasound catheter.

One embodiment of the present invention comprises an ultrasound catheter configured to be advanced into a patient's neurovascular system. The catheter includes an elongate outer sheath and an elongate hollow inner core. The elongate outer sheath defines a central lumen that extends longitudinally from an outer sheath proximal region to an outer sheath distal region. The elongate hollow inner core is positioned in the central lumen. The inner core defines a utility lumen configured to receive a guidewire. The inner core has a distal region that terminates at a point that is proximal to the outer sheath distal region. The inner core comprises a reinforcing member that extends along at least a portion of the inner core. The reinforcing member is configured to reduce ovalization of the inner core as the catheter is bent. A tubular inner support member is coupled to the inner core distal region. A tubular outer support member is coupled to the outer sheath distal region. An ultrasound radiating member has an inner passage. The ultrasound radiating member is positioned generally the between the inner and outer support members, such that the inner support member passes through the hollow inner core and the outer support member is positioned over an outer surface of the ultrasound radiating member.

Another embodiment of the invention comprises an neurovascular catheter. The catheter includes a tubular body having a proximal end and a distal end. The tubular body comprises an inner tubular component and an outer tubular component. The outer tubular component has a proximal region, a distal region end and a lumen extending therethrough. The inner tubular component is positioned within the lumen of the outer tubular component and extends from the proximal region to distal region of the outer tubular component. The inner tubular component forms, at least in part a utility lumen, that extends from proximal end of the tubular body to the distal end of the tubular body. The inner tubular body is formed at least in part from a composite tube comprising an inner member. A reinforcing coil surrounds the inner member, and an outer member covers the reinforcing coil. At least one ultrasound radiating member is positioned generally between the outer tubular component and the inner tubular component at the distal end of the tubular body. A least one electrical wire is operatively connected to the ultrasound radiating member. The at least one electrical wire extends at least partially through a space between the outer tubular component and the inner tubular component.

Another embodiment of the invention comprises a catheter having a distal end and a proximal end. The catheter comprises an elongate outer sheath and an enlogate inner sheath. The elongate outer sheath has an exterior surface. The distal end portion of said outer sheath has an outer diameter of less than about 5 French for advancement through a small blood vessel. The outer sheath defines a central lumen extending longitudinally therethrough. An elongate inner core extends through said central lumen of said outer sheath and terminates at an exit port located at the distal end of the catheter. The inner core defines a utility lumen adapted to receive a guidewire lumen. An ultrasound member is positioned at the distal end of the catheter body generally between the outer sheath and the inner core. A guidewire is configured to be slideably received within the utility lumen for advancement of the catheter to a treatment site. The guidewire having a diameter that is less than or equal to about 0.017 inches. The catheter is configured such that the catheter can be subjected to a 180 degree bend having a radius of less than about 10 mm while still permitting the catheter to slide over the guidewire.

In one embodiment of the present invention, a method of manufacturing an ultrasound catheter comprises providing an elongate outer sheath that defines a central lumen extending longitudinally from an outer sheath proximal region to an outer sheath distal region. The method further comprises providing a plurality of elongate electrical conductors within the central lumen. The method further comprises positioning an elongate inner core in the central lumen, such that the plurality of electrical conductors are positioned between the inner core and the outer sheath. The method further comprises coupling a tubular inner support member to a distal region of the elongate inner core. The method further comprises mounting an ultrasound radiating member to the inner support member. The ultrasound radiating member includes a hollow inner core through which the inner support member is positioned. The method further comprises coupling a tubular outer support member to a distal region of the elongate outer sheath. The tubular outer support member is positioned over an outer surface of the ultrasound radiating member.

### BRIEF DESCRIPTION OF THE DRAWINGS

FIG. 1 is a side view of an ultrasound catheter that is particularly well suited for insertion into small blood vessels of the human body.

FIG. 2A is a cross-sectional view of a distal end of the ultrasound catheter of FIG. 1.

FIG. 2B is a cross-sectional view of the ultrasound catheter of FIG. 1 taken through line 2B-2B of FIG. 2A.

FIG. 3 is a fragmentary cross-sectional view of a catheter section having a multi-layered section with a variable flexibility.

FIG. 4 is a fragmentary cross-sectional view of a catheter section having a multi-layered section with a substantially constant flexibility.

FIG. 5 is a fragmentary cross-sectional view of a catheter section having a multi-layered section with a partial spiral cut.

FIG. 6 is a cross-sectional view of the interface between a catheter distal end and a catheter junction section.

FIG. 7A is a partial cutaway of a distal section of a catheter comprising a woven, braided kink-resisting member.

FIG. 7B is a partial cutaway of a distal section of a catheter comprising a helically would coil kink-resisting member.

FIG. 8 is an exploded view of the components comprising an ultrasound catheter distal end and junction section.

FIG. 9 is a side view of a catheter having a variable flexibility.

FIG. 10 is a radial cross-sectional view of a distal portion of a catheter having variable diameter stiffener strands and a variable outer diameter.

FIG. 11 is a radial cross-sectional view of a midsection of a catheter having variable diameter stiffener strands and a variable outer diameter.

FIG. 12 is a radial cross-sectional view of a proximal portion of a catheter having variable diameter stiffener strands and a variable outer diameter.

FIG. 13 is a radial cross-sectional view of a distal portion of a catheter having variable diameter stiffener strands and a substantially constant outer diameter.

FIG. 14 is a radial cross-sectional view of a midsection of a catheter having variable diameter stiffener strands and a substantially constant outer diameter.

FIG. 15 is a radial cross-sectional view of a proximal portion of a catheter having variable diameter stiffener strands and a substantially constant outer diameter.

FIG. 16 is a radial cross-sectional view of a distal portion of a catheter having a variable thickness inner stiffener layer.

FIG. 17 is a radial cross-sectional view of a midsection of a catheter having a variable thickness inner stiffener layer.

FIG. 18 is a radial cross-sectional view of a proximal portion of a catheter having a variable thickness inner stiffener layer.

FIG. 19 is a longitudinal cross-sectional view of a catheter 100 corresponding to the three radial cross sections of FIGS. 16 through 18.

FIG. 20 is a longitudinal cross-sectional view of a catheter having a non-discretely gradually increasing stiffness proximally.

FIG. 21 is a radial cross-sectional view of a distal portion of a catheter manufactured by stretching an extruded tubular member having a substantially constant cross-sectional configuration, a first outer sheath material and a second stiffener strand material.

FIG. 22 is a radial cross-sectional view of a midsection of a catheter manufactured by stretching an extruded tubular member having a substantially constant cross-sectional configuration, a first outer sheath material and a second stiffener strand material.

FIG. 23 is a radial cross-sectional view of a proximal portion of a catheter manufactured by stretching an extruded tubular member having a substantially constant cross-sectional configuration, a first outer sheath material and a second stiffener strand material.

FIG. 24 is a cross-sectional view of a catheter having a coaxial segmented structure.

FIG. 25 is an exemplary graph illustrating relative catheter flexibility as a function of axial catheter position.

FIG. 26A is a partial cutaway side view of a composite tubular body with improved flexibility and kink- and buckle-resistance.

FIG. 26B is a cross-sectional view of the catheter of FIG. 26A taken along line 26B-26B.

FIG. 27 is a cut-away view of selected internal components of a backend hub configured for use with the composite tubular body of FIGS. 26A and 26B.

FIG. 28 is a cross-sectional view of the distal end of an ultrasound catheter that includes the composite tubular body of FIGS. 26A and 26B.

FIG. 29 is a cross-sectional view of a modified embodiment the distal end of an ultrasound catheter.

### DETAILED DESCRIPTION OF PREFERRED EMBODIMENTS

Introduction.

The advancement of an ultrasound catheter through a blood vessel to a treatment site can be difficult and dangerous, particularly when the treatment site is located within a small vessel in the distal region of a patient's vasculature. Accessing the treatment site may involve navigating a tortuous path around difficult bends and turns, such as the main and subsequent branches of the middle cerebral artery. During advancement through the vasculature, bending resistance along the distal end portion of the catheter can limit the ability of the catheter to make small radius turns. Moreover, as the catheter is advanced, the distal tip of the catheter is often in contact with the inner wall of the blood vessel. The stiffness and rigidity of the distal tip of the catheter may lead to significant trauma or damage to the tissue along the inner wall of the blood vessel. As a result, advancement of an ultrasound catheter through small blood vessels can be extremely hazardous. Therefore, an improved ultrasound catheter design having variable flexibility and/or stiffness along the length of the catheter body will allow a physician to more easily navigate difficult turns in small blood vessels while reducing trauma and/or damage along the inner walls of the blood vessels.

Certain embodiments described herein provide an ultrasound catheter that is well suited for use in the treatment of small blood vessels or other body lumens having a small inner diameter. Such embodiments can be used to enhance the therapeutic effects of drugs, medication, pharmacological agents and other therapeutic compounds at a treatment site within the body. See, for example, U.S. Pat. Nos. 5,318,014; 5,362,309; 5,474,531; 5,628,728; 6,001,069; and 6,210,356. Certain embodiments described herein are particularly well suited for use in the treatment of thrombotic occlusions in small blood vessels, such as, for example, the cerebral arteries. In addition, certain embodiments described herein can be used in other therapeutic applications, such as, for example, performing gene therapy (see, for example, U.S. Pat. No. 6,135,976), activating light activated drugs for producing targeted tissue death (see, for example, U.S. Pat. No. 6,176,842) and causing cavitation and/or controlled cavitation to produce various desirable biological effects (see, for example, U.S. Pat. No. RE36,939). Moreover, such therapeutic applications can be used in wide variety of locations within the body, such as, for example, in other parts of the circulatory system, in solid tissues, in duct systems and in body cavities. The ultrasound catheters disclosed herein, and variations thereof, can be used in other medical applications, such as, for example, diagnostic and imaging applications. The contents of the patents referenced above are hereby incorporated by reference herein.

Ultrasound catheters and methods disclosed herein, and similar variations thereof, can also be used in applications wherein the ultrasonic energy provides a therapeutic effect by itself. For example, ultrasonic energy can be effective in preventing and/or reducing stenosis and/or restenosis; causing tissue ablation, abrasion or disruption; promoting temporary or permanent physiological changes in intracellular or intercellular structures; and rupturing micro-balloons or microbubbles for drug delivery. See, for example, U.S. Pat. Nos. 5,269,291 and 5,431,663, which are hereby incorporated by reference herein. In addition, the methods and devices disclosed herein can also be used in applications that do not require the use of a catheter. For example, the methods and devices disclosed herein can be used to enhance hyperthermic drug treatment or to cause transdermal enhancement of the therapeutic effects of drugs, medication, pharmacological agents, or other therapeutic compounds at a specific site within the body. The methods and devices disclosed herein can also be used to provide a therapeutic or diagnostic effect without the use of a therapeutic compound. See, for example, U.S. Pat. Nos. 4,821,740; 4,953,565; 5,007,438 and 6,096,000, the contents which are hereby incorporated by reference herein.

As used herein, the term “ultrasonic energy” is used broadly, and includes its ordinary meaning, and further includes mechanical energy transferred through pressure or compression waves with a frequency greater than about 20 kHz. In one embodiment, the waves of the ultrasonic energy have a frequency between about 500 kHz and about 20 MHz, and in another embodiment the waves of ultrasonic energy have a frequency between about 1 MHz and about 3 MHz. In yet another embodiment, the waves of ultrasonic energy have a frequency of about 3 MHz.

As used herein, the term “catheter” is used broadly, and include its ordinary meaning, and further includes an elongate flexible tube configured to be inserted into the body of a patient, such as, for example, a body cavity, duct or vessel.

As used herein, the term “therapeutic compound” refers broadly, in addition to its ordinary meaning, to a drug, medicament, dissolution compound, genetic material, or any other substance capable of effecting physiological functions. Additionally, any mixture comprising any such substances is encompassed within this definition of “therapeutic compound”.

As used herein, the term “end” refers, in addition to its ordinary meaning, to a region, such that “proximal end” includes “proximal region”, and “distal end” includes “distal region”.

As used herein, the term “proximal element joint” refers generally, and in addition to its ordinary meaning, to a region where a proximal portion of an ultrasound radiating member is attached to other components of an ultrasound catheter.

Exemplary embodiments of an ultrasound, drug delivery catheter.

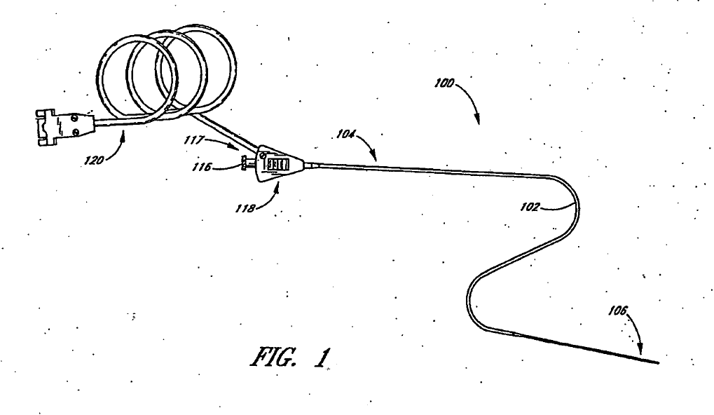

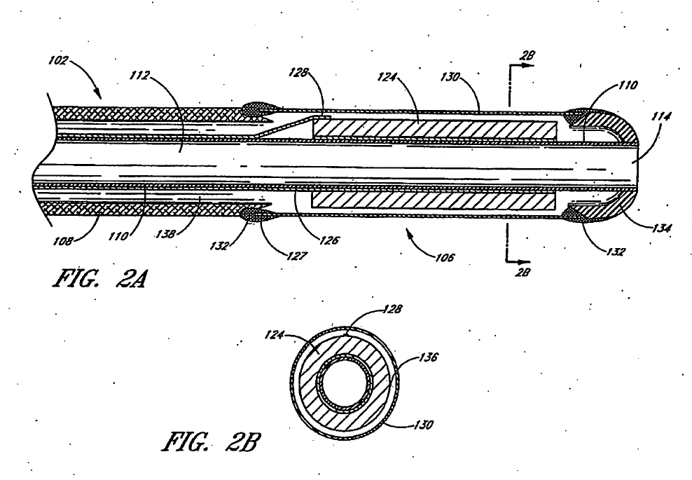

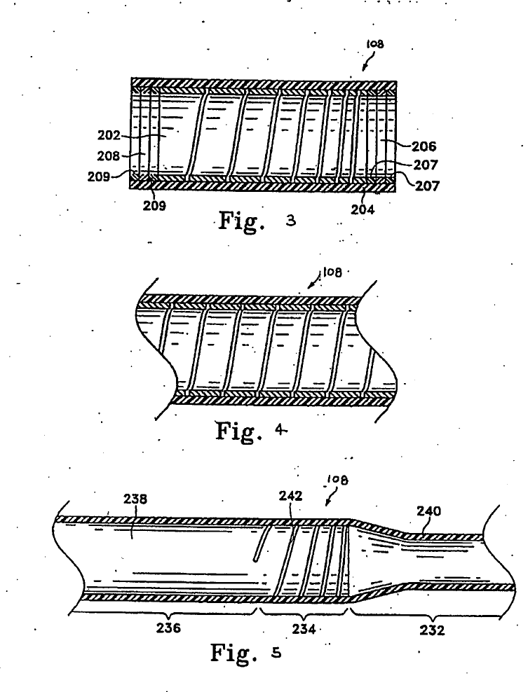

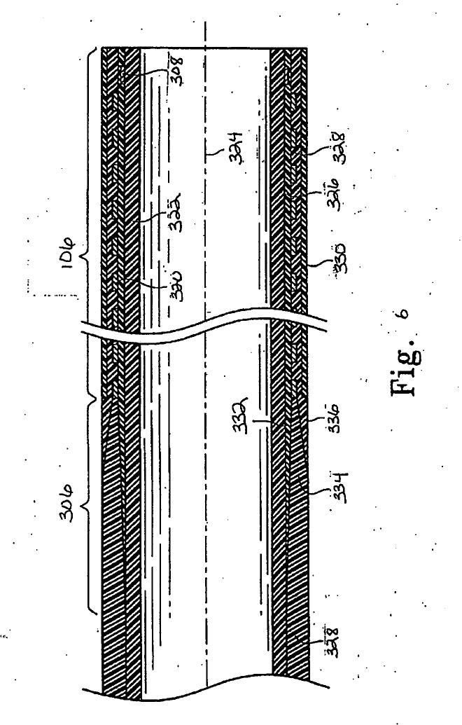

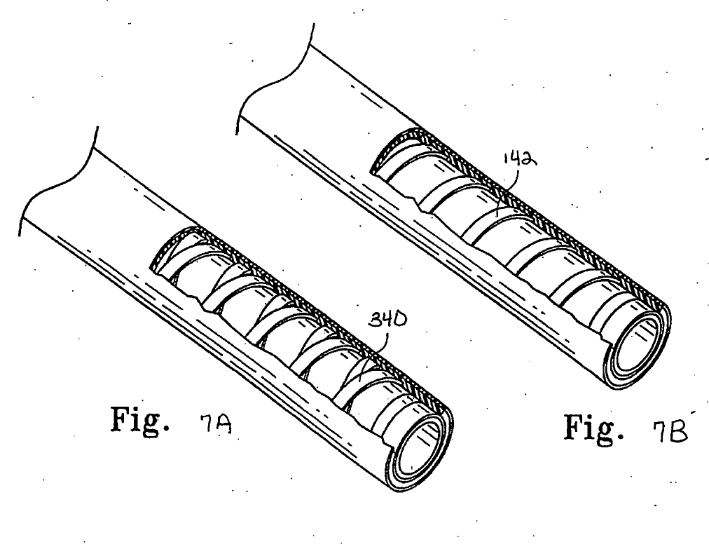

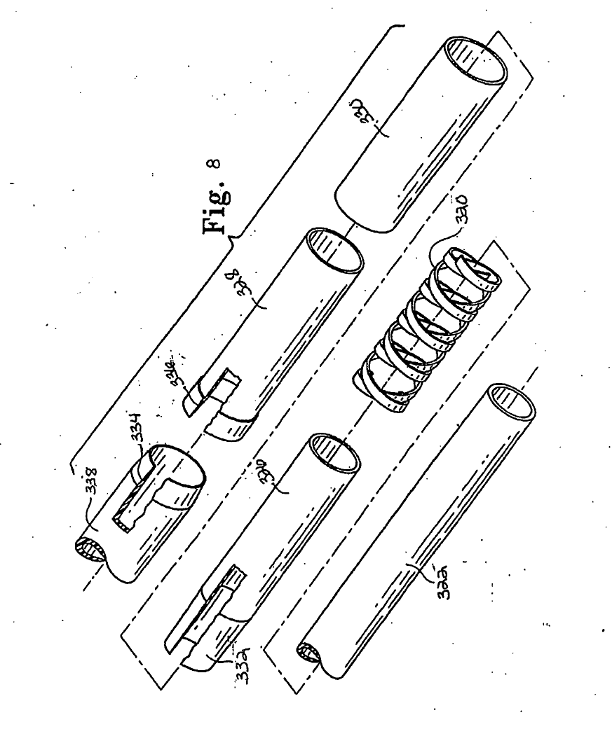

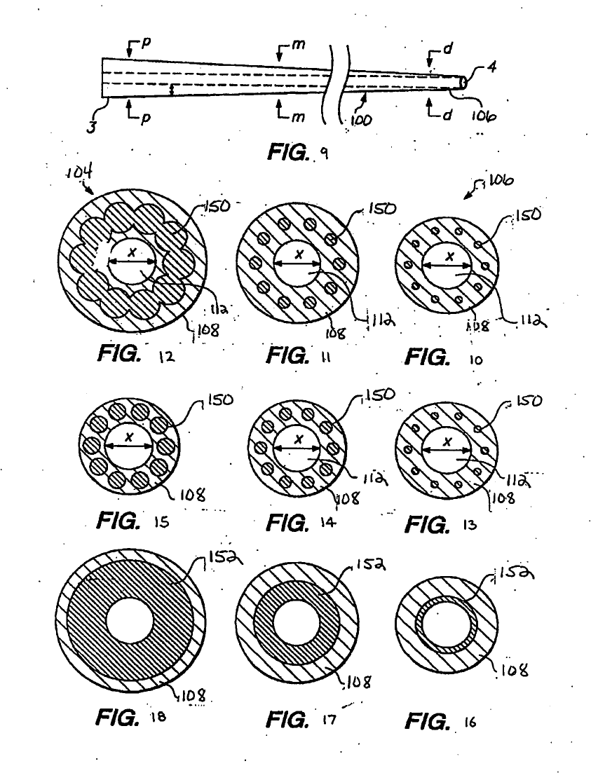

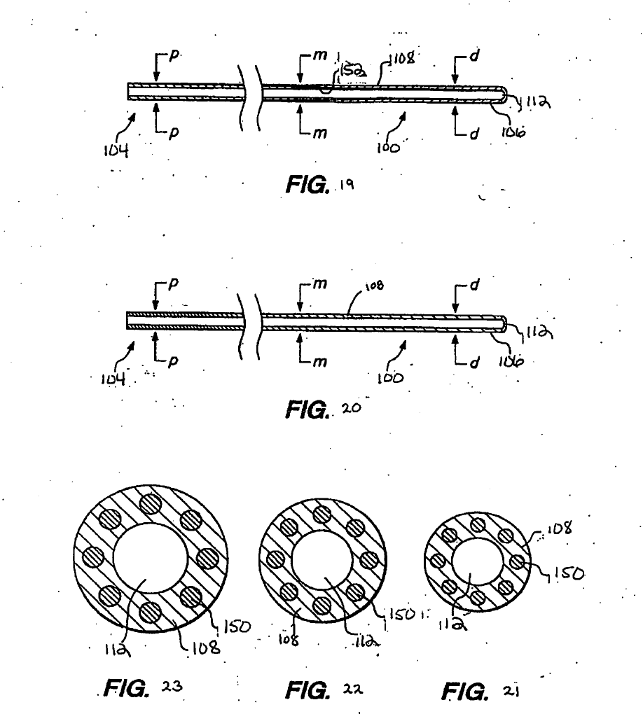

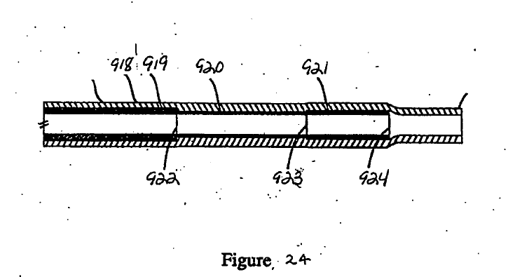

FIGS. 1 through 2B illustrated an exemplary embodiment of an ultrasound catheter 100 that is well suited for use within small vessels of the distal anatomy, such as the remote, small diameter blood vessels located in the brain.

As shown in FIG. 1 and 2A, the ultrasound catheter 100 generally comprises a multi-component tubular body 102 having a proximal end 104 and a distal end 106. The tubular body 102 and other components of the catheter 100 can be manufactured in accordance with any of a variety of techniques well known in the catheter manufacturing field. As discussed in more detail below, suitable material dimensions can be readily selected taking into account the natural and anatomical dimensions of the treatment site and of the desired percutaneous access site.

The tubular body 102 can be divided into multiple sections of varying stiffness. For example, a first section, which includes the proximal end 104, is generally more stiff than a second section, which lies between the proximal end 104 and the distal end 106 of the catheter. This arrangement facilitates the movement and placement of the catheter 102 within small vessels. A third section, which includes at least one ultrasound radiating member 124, is generally stiffer than the second section due to the presence of the ultrasound radiating member 124.

In the exemplary embodiments described herein, the assembled ultrasound catheter 100 has sufficient structural integrity, or “pushability,” to permit the catheter to be advanced through a patient's vasculature to a treatment site without significant buckling or kinking. In addition, the catheter can transmit torque (that is, the catheter has “torqueability”), thereby allowing the distal portion of the catheter to be rotated into a desired orientation by applying a torque to the proximal end 104.

Referring now to FIG. 2A, the elongate flexible tubular body 102 comprises an outer sheath 108 positioned upon an inner core 110. In an embodiment particularly well suited for small vessels, the outer sheath 108 comprises a material such as extruded Pebax®, polytetrafluoroethylene (“PTFE”), PEEK, PE, polyimides, braided polyimides and/or other similar materials. The distal end portion of the outer sheath 108 is adapted for advancement through vessels having a small diameter, such as found in the brain. In an exemplary embodiment, the distal end portion of the outer sheath 108 has an outer diameter between about 2 French and about 5 French. In another exemplary embodiment, the distal end portion of the outer sheath 108 has an outer diameter of about 2.8 French. In an exemplary embodiment, the outer sheath 108 has an axial length of approximately 150 centimeters. In other embodiments, other dimensions can be used.

In other embodiments, the outer sheath 108 can be formed from a braided and/or coiled tubing comprising, for example, high or low density polyethylenes, urethanes, nylons, and so forth. Such a configuration enhances the flexibility of the tubular body 102. For enhanced pushability and torqueability, the outer sheath 108 can be formed with a variable stiffness from the proximal to the distal end. To achieve this, a stiffening member can be included along the proximal end of the tubular body 102. In one exemplary embodiment, the pushability and flexibility of the tubular body 102 are controlled by manipulating the material and thickness of the tubular body 102, while the torqueability, kink resistance, distortion (also referred to as “ovalization”) and burst strength of the tubular body 102 are controlled by incorporation of braiding and/or coiling along or into the tubular body 102.

In one particular embodiment, the outer tubular member 108 comprises a PTFE layer that surrounds a Teflon inner layer. As mentioned above, the outer tubular member 108 generally tapers from the proximal end to the distal end. In one embodiment, the proximal end of the outer member is reinforced with reinforcement member (e.g., a stainless steel flat wire coil) positioned between the PTFE and Teflon layers. A Tensile fiber (e.g., Kevlar or Vectron) may also be positioned between the layers to add tensile strength to the catheter.

The inner core 110 at least partially defines a delivery lumen 112. In an exemplary embodiment, the delivery lumen 112 extends longitudinally along substantially the entire length of the catheter 100. The delivery lumen 112 comprises a distal exit port 114 and a proximal access port 116. Referring again to FIG. 1, the proximal access port 116 is defined by therapeutic compound inlet port 117 of back end hub 118, which is attached to the proximal end 104 of the other sheath 108. In an exemplary embodiment, the illustrated back end hub 118 is attached to a control box connector 120, which will be described in more detail below. In a modified embodiment, electronics and/or control circuitry for operating the ultrasound assembly are incorporated into the back end hub 118.

In an exemplary embodiment, the delivery lumen 112 is configured to receive a guide wire (not shown). In one embodiment, the guidewire has a diameter of approximately 0.008 inches to approximately 0.012 inches. In another embodiment, the guidewire has a diameter of about 0.010 inches. In an exemplary embodiment, the inner core 110 comprises polyimide or a similar material which, in some embodiments, can be braided and/or coiled to increase the flexibility of the tubular body 102.

Referring now to the exemplary embodiment illustrated in FIGS. 2A and 2B, the distal end 106 of the tubular body 102 comprises an ultrasound radiating member 124. The ultrasound radiating member 124 can comprise an ultrasound transducer that converts, for example, electrical energy into ultrasonic energy. In a modified embodiment, the ultrasonic energy can be generated by an ultrasound transducer that is remote from the ultrasound radiating element 124, and the ultrasonic energy can be transmitted via, for example, a wire to the ultrasound radiating member 124.

As illustrated in FIGS. 2A and 2B, the ultrasound radiating member 124 is configured as a hollow cylinder. As such, the inner core 110 can extend through the hollow core of the ultrasound radiating member 124. The ultrasound radiating member 124 can be secured to the inner core 110 in any suitable manner, such as with an adhesive. A potting material can also be used to further secure the ultrasound radiating member 124 to the central core.

In other embodiments, the ultrasound radiating member 124 has different shape. For example, the ultrasound radiating member 124 can be shaped as a solid rod, a disk, a solid rectangle or a thin block. In still other embodiments, the ultrasound radiating member 124 comprises a plurality of smaller ultrasound radiating elements. The embodiments illustrated in FIGS. 1 through 2B advantageously provide enhanced cooling of the ultrasound radiating member 124. For example, in an exemplary embodiment, a therapeutic compound is delivered through the delivery lumen 112. As the therapeutic compound passes through the lumen of the ultrasound radiating member 124, the therapeutic compound advantageously removes heat generated by the ultrasound radiating member 124. In another embodiment, a return path can be formed in region 138 between the outer sheath 108 and the inner core 110 such that coolant from a coolant system can be directed through region 138.

In an exemplary embodiment, the ultrasound radiating member 124 is selected to produce ultrasonic energy in a frequency range adapted for a particular application. Suitable frequencies of ultrasonic energy for the applications described herein include, but are not limited to, from about 20 kHz to about 20 MHz. In one embodiment, the frequency is between about 500 kHz and about 20 MHz, and in another embodiment, the frequency is between about 1 MHz and about 3 MHz. In yet another embodiment, the ultrasonic energy has a frequency of about 3 MHz.

For example, in one embodiment, the dimensions of the ultrasound radiating member are selected to provide a ultrasound radiating member that is capable of generating sufficient acoustic energy to enhance lysis without significantly adversely affecting catheter maneuverability.

As described above, in the embodiment illustrated in FIGS. 1 through 2B, ultrasonic energy is generated from electrical power supplied to the ultrasound radiating member 124. The electrical power can be supplied through control box connector 120, which is connected to conductive wires 126, 128 that extend through the tubular body 102. In another embodiment, the electrical power can be supplied from a power supply contained within the back end hub 118. The conductive wires 126, 128 can be secured to the inner core 110, can lay along the inner core 110, and/or can extend freely in the region 138 between the inner core 110 and the outer sheath 108. In the illustrated embodiments, the first wire 126 is connected to the hollow center of the ultrasound radiating member 124, while the second wire 128 is connected to the outer periphery of the ultrasound radiating member 124. In an exemplary embodiment, the ultrasound radiating member 124 comprises a transducer formed of a piezoelectric ceramic oscillator or a similar material.

In the exemplary embodiment illustrated in FIGS. 2A and 2B, the distal end 106 of the catheter 100 includes a sleeve 130 that is generally positioned about the ultrasound radiating member 124. In such embodiments, the sleeve 130 comprises a material that readily transmits ultrasonic energy. Suitable materials for the sleeve 130 include, but are not limited to, polyolefins, polyimides, polyesters and other materials that readily transmit ultrasonic energy with minimal absorption of the ultrasonic energy. The proximal end of the sleeve 130 can be attached to the outer sheath 108 with an adhesive 132. In certain embodiments, to improve the bonding of the adhesive 132 to the outer sheath 108, a shoulder 127 or notch is formed in the outer sheath 108 for attachment of the adhesive 132 thereto. In an exemplary embodiment, the outer sheath 108 and the sleeve 130 have substantially the same outer diameter. In other embodiments, the sleeve 130 can be attached to the outer sheath 108 using heat bonding techniques, such as radiofrequency welding, hot air bonding, or direct contact heat bonding. In still other embodiments, techniques such as over molding, dip coating, film casting and so forth can be used.

Still referring to the exemplary embodiment illustrated in FIGS. 2A and 2B, the distal end of the sleeve 130 is attached to a tip 134. As illustrated, the tip 134 is attached to the distal end of the inner core 110. In one embodiment, the tip is between about 0.5 millimeters and about 4.0 millimeters long. In another embodiment, the tip is about 2.0 millimeters long. As illustrated, in certain embodiments the tip is rounded in shape to reduce trauma or damage to tissue along the inner wall of a blood vessel or other body structure during advancement toward a treatment site.

As illustrated in FIG. 2B, the catheter 100 can include at least one temperature sensor 136 along the distal end 106. In one embodiment, the temperature sensor 136 is positioned on or near the ultrasound radiating member 124. Suitable temperature sensors include but are not limited to, diodes, thermistors, thermocouples, resistance temperature detectors, and fiber optic temperature sensors that used thermalchromic liquid crystals. In an exemplary embodiment, the temperature sensor 136 is operatively connected to a control box (not shown) through a control wire that extends through the tubular body 102 and back end hub 118, and that is operatively connected to the control box via control box connector 120. The control box preferably includes a feedback control system having the ability to monitor and control the power, voltage, current and phase supplied to the ultrasound radiating member 124. In this manner, the temperature along the relevant region of the catheter 100 can be monitored and controlled. Details of the control box can be found in Assignee's co-pending U.S. patent applications Ser. Nos. 10/309,388 and 10/309,417, which are both incorporated by reference herein in their entirety.

In embodiments wherein multiple ultrasound radiating members are positioned in the catheter distal region, a plurality of temperature sensors can be positioned adjacent to the ultrasound radiating members. For example, in one such embodiment, a temperature sensor is positioned on or near each of the multiple ultrasound radiating members.

Exemplary Embodiments of Use

In an exemplary method, the ultrasound catheter 100 can be used to remove an occlusion from a small blood vessel. In such an exemplary application, a free end of a guidewire is percutaneously inserted into a patient's vasculature at a suitable first puncture site. The guidewire is advanced through the vasculature toward a treatment site where the blood vessel is occluded by a thrombus. In one embodiment, the guidewire wire is directed through the thrombus, and is left in the thrombus during treatment to aid in dispersion of the therapeutic compound into the thrombus.

After advancing the guidewire to the treatment site, the catheter 100 is percutaneously inserted into the patient's vasculature through the first puncture site, and is advanced along the guidewire towards the treatment site using conventional over-the-guidewire techniques. The catheter 100 is advanced until the distal end 106 is positioned at or within the occlusion. In a modified embodiment, the distal end 106 comprises one or more radiopaque markers (not shown) to aid in positioning the distal end 106 within the treatment site.

After the catheter is positioned, the guidewire can be withdrawn from the delivery lumen 112. A therapeutic compound source (not shown), such as a syringe with a Luer fitting, is hydraulically connected to the therapeutic compound inlet port 117 and the control box connector 120 is connected to the control box. Thus, a therapeutic compound can be delivered through the delivery lumen 112 and out the distal exit port 114 to the occlusion. One exemplary therapeutic compound appropriate for treating a thrombus is an aqueous solution containing heparin, urokinase, streptokinase, and/or tissue plasminogen activator.

The ultrasound radiating member 124 can be activated to emit ultrasonic energy from the distal end 106 of the catheter 100. As described above, suitable frequencies for the ultrasonic energy include, but are not limited to, from about 20 kHz to about 20 MHz. In one embodiment, the frequency is between about 500 kHz and about 20 MHz, and in another embodiment the frequency is between about 1 MHz and 3 MHz. In yet another embodiment, the ultrasonic energy has a frequency of about 3 MHz. The therapeutic compound and ultrasonic energy are applied until the thrombus is partially or entirely dissolved. Once the thrombus has been dissolved sufficiently, the catheter 100 is withdrawn from the treatment site.

Methods of manufacture.

The catheters described herein can be manufactured by sequentially positioning the various catheter components onto the catheter assembly. For example, in one method of manufacture, the ultrasound radiating member 124 is positioned around the outer surface of an intermediate portion of an elongate tube. The elongate tube serves as the inner core 110 and defines delivery lumen 112. The first and second wires 126, 128 are then also disposed along the outer surface of the inner core 110 proximal to the ultrasound radiating member 124. The first wire 126 is electrically connected to an inner surface of the ultrasound radiating member 124, and the second wire is electrically connected to an outer surface of the ultrasound radiating member 124, as illustrated in FIG. 2A. The electrical connections can be accomplished using, for example, a solder joint.

After the ultrasound radiating member 124 and wires 126, 128 are secured to the inner core 110, an outer sheath 108 is positioned over a portion of the inner core, leaving the ultrasound radiating member 124 uncovered by the outer sheath 108, as illustrated in FIG. 2A. A cylindrical sleeve 130 is then positioned over the ultrasound radiating member 124, and is secured to the distal end of the outer sheath 108 with an adhesive 132. A rounded distal tip 134 can then be secured to the sleeve 130 and the inner core 110, and any excess length of the elongate tube extending distal to the distal tip 134 can be removed.

Although an exemplary catheter manufacturing technique has been expounded above, other manufacturing techniques can be used, additional components can be included, and the components set forth above can be modified. For example, in certain embodiments, the catheter 100 further comprises a temperature sensor 136 positioned near the ultrasound radiating member 124, as described above. In other embodiments, the outer sheath 108 can be modified to manipulate the flexibility of the catheter 100, such as by including a stiffening component or metallic braiding and/or coiling.

Techniques to reduce buckling or kinking in an ultrasound, drug delivery catheter.

As described above, the ultrasound catheter should have sufficient structural integrity, or “pushability,” to permit the catheter to be advanced through a patient's vasculature to a treatment site without buckling or kinking. Buckling and kinking can obstruct the delivery lumen and cause excessive friction between the catheter and the blood vessel. In this section, several techniques are described for reducing the likelihood of buckling and kinking of the catheter with a minimal increase in the catheter stiffness by disposing a spirally cut thin polymeric tubing in regions the catheter body susceptible to buckling or kinking. Such regions may in the region of the proximally adjacent the ultrasound element.

FIG. 3 illustrates a partial section of an outer sheath 108 (or inner core 110) that may be located proximal to the location of the ultrasound radiating member, but that is still in a distal region of the catheter that is susceptible to buckling or kinking. As illustrated, in such embodiments, the outer sheath 108 may comprise an inner helically cut polymeric inner tubing stiffener member 202 and an outer polymeric layer 204.

In an exemplary embodiment, the inner tubing stiffener member 202 comprises a simple section of tubing that has been spirally cut from its inner surface to its outer surface as shown in FIG. 3. The spiral cut shown in FIG. 3 decreases in pitch in the distal direction to provide for a varying amount of flexibility in the distal direction. After it has been cut, the inner tubing stiffener member 202 is slightly stretched to provide increased flexibility. The inner tubing stiffener member 202 can comprise a wide variety of materials, such as linear low density polyethylene (“LLDPE”) or low density polyethylene (“LDPE”). In certain embodiments, the inner tubing stiffener member 202 further comprises a small amount of ethylene vinyl acetate (“EVA”).

In an exemplary embodiment, the wall thickness of the outer sheath 108 is between approximately 0.005 inches and approximately 0.002 inches. In another embodiment, the wall thickness of the outer sheath 108 is approximately 0.0015 inches. The pitch of the cut in the inner tubing stiffener member 202 can be of any appropriate length. In a modified embodiment, the pitch of the cut in the inner tubing stiffener member 202 is variable, thereby providing a section of varying flexibility.

The outer polymeric layer 204 can comprise any of a wide variety of materials. Such materials include, but are not limited to, Pebax®, PTFE, PEEK, PE, polyurethanes, polyvinyl chloride, LDPE, LLDPE, or mixtures thereof. In an exemplary embodiment, the outer polymeric layer 204 comprises a heat shrinkable tubing of LDPE or LLDPE, having an EVA content of at least 10% EVA. In another embodiment, the EVA content is between approximately 12% and approximately 20%. In another embodiment, the outer polymeric layer thickness is between approximately 0.005 inches and approximately 0.010 inches. In still another embodiment, the outer polymeric layer thickness is approximately 0.003 inches. The aforementioned polymers can be cross-linked by radiation to increase their strength and to promote heat shrinking.

The outer sheath illustrated in FIG. 3 can be manufactured using a variety of techniques, including the following exemplary technique. A distal spacer 207 is placed on a mandrel of an appropriate size adjacent the inner stiffener member 202. A proximal spacer 209 is also placed on the mandrel. An adhesive such as thermoplastic can be applied to the outside of this assemblage but is not required. A heat shrinkable outer polymeric layer 204 is positioned over the assemblage previously placed on the interior mandrel. The heat shrinkable outer polymeric layer 204 is then heat shrunk onto the assemblage. In an exemplary embodiment, the material comprising the inner stiffener member 202 has a melt temperature in the region of that of the heat shrink temperature of the heat shrinkable outer polymeric layer 204. This creates a unitary structure having a high kink resistance and variable flexibility and pushability.

The outer polymeric layer 204 in FIG. 3 can also be applied by dipping the inner stiffener member 202 into a molten polymer bath or into a polymer dissolved in a solution or into a suspension of latex comprising the outer layer polymer. The outer polymeric layer 204 can also be placed on the inner stiffener member 202 by spraying or otherwise applying the material. The catheters and catheter sections described herein can be coated or otherwise treated both inside and outside to increase their lubricity.

FIG. 4 illustrates a modified outer sheath 108. In such embodiments, the spirally cut pitch is substantially constant, but otherwise the section is identical to that described in connection with FIG. 3. This modified embodiment provides kink resistance with enhanced flexibility.

FIG. 5 illustrates a modified outer sheath 108 in which the spiral cut 242 is formed on only a portion of the interior stiffener member. In this embodiment, the spiral cut 242 provides an intermediate portion 234 having variable flexibility, positioned between a smaller diameter distal portion 232 and a larger diameter proximal portion 236.

The outer sheath illustrated in FIG. 5 can be manufactured using a variety of techniques, including the following exemplary technique. The inner stiffener member 238 comprises a polymer relatively stiffer than the outer polymeric layer 240. In such embodiments, the inner stiffener member 238 comprises a polymer such as polypropylene, high density polyethylene (“HDPE”), polyimides, polyamides (many of the nylons), and some of the stiffer grades of polyethylene such as LLDPE and LDPE. The spiral cut 242 extends from the outer surface of the inner stiffener member 238 to the inner surface of the inner stiffener member 238. In an exemplary embodiment, the spiral cut 242 is slightly expanded to provide a small gap therein. In such embodiments, the spiral cut 242 stops at the proximal end of the intermediate portion 234.

In one embodiment, the outer polymeric layer 240 comprises a, heat-shrinkable material such as a polyethylene. Other suitable materials for the outer polymeric layer 240 include polyurethane, polyvinyl chloride, and other softer and more compliant materials. In such embodiments, the outer polymeric layer 240 extends from the proximal end of the catheter to the distal end of the catheter 230.

The embodiment illustrated in FIG. 5 has a variety of advantages, including ease of construction. For instance, the stiffener member 238 provides a proximal portion 238 and an intermediate portion 234 that are easy to push and that retain pushability while having less stiffness than more proximal catheter portions. The specific pattern of the spiral cut 242 in the inner stiffener member 238 provides a smoother transition in stiffness between the proximal portion 236 and the distal portion 232 than does a section of tubing having an intermediate stiffness.

Braided catheter.

As described above, and as illustrated in FIG. 1, certain ultrasound catheter embodiments have a relatively more flexible distal end 106 and a relatively less flexible proximal end 104. Such an arrangement increases catheter maneuverability to facilitate navigation of the catheter through the small vessels of a patient's vasculature. In particular, the relatively less flexible proximal end 104 is able to effectively transmit torquing and pushing operations during placement of the catheter. The ultrasound catheter 100 proximal end 104 and distal end 106 are joined at an intermediate junction section 306, which is illustrated in greater detail in FIG. 6. Junction section 306 has an intermediate flexibility, and, although forming only a small percentage of the overall length of the catheter, is nevertheless long when compared to the catheter diameter.

As illustrated in FIG. 6, in an exemplary embodiment, the catheter distal end 106 comprises a number of polymer layers and a kink-resisting member 320. In certain embodiments, the catheter distal end 106 further comprises a radiopaque band 308. The kink-resisting member 320 extends along at least a portion of the distal end 106. The proximal end of kink-resisting member 320 is also the proximal most extent of the distal end 106. Kink-resisting member 320 can comprise a woven braid 340 (as illustrated in FIG. 7A) or can comprise an un-woven braid. The kink-resisting member 320 can also comprise a helically wound coil 342, as illustrated in FIG. 7B. The woven braid 340 can comprise, for example, a pair of counter-woven helically wound coils such as described as a non-woven braid. Kink-resisting member 320 can comprise ribbons, wires, individual fibers, accumulated fibers, woven fibers, or some combination thereof. As used herein, “ribbon”, in addition to its ordinary meaning, further refers to an elongate element having a cross-section that is rectangular, oval or semi-oval. In an exemplary embodiment using the super-elastic alloys described below, particularly those containing nickel and titanium, the thickness of a ribbon used in the kink-resisting member 320 is between approximately 0.00025 inches and approximately 0.0025 inches, and the width a ribbon used in the kink-resisting member 320 is between approximately 0.001 inches and approximately 0.010 inches. In an exemplary embodiment using metallic wire in the kink-resisting member 320, the metallic wire has a diameter between approximately 0.00020 inches and approximately 0.002 inches. Generally, ribbons comprising suitable polymers, such as liquid crystal polymers (“LCP”), are of a size similar to those for super-elastic alloys.

As noted above, the kink-resisting member 320 can comprise a super-elastic alloy such as titanium/nickel materials known as nitinol. Commercial nitinol alloys containing up to about 8% or more of one or more of the members of the iron group of the periodic table are considered to be encompassed within the class of super-elastic nickel/titanium alloys.

In certain embodiments where a super-elastic alloy is used to form the kink-resisting member 320, after a braid has been woven using a plurality of members, a heat treatment is applied to the kink-resisting member. The heat treatment reduces the likelihood that the braid will unravel during subsequent handling or will change in diameter or spacing during that handling. In the heat treatment, the braids are placed on a heat-resistant mandrel, for example by weaving them onto that mandrel, and the mandrel in then placed in an oven at a elevated temperature for a few minutes. In one embodiment, the oven temperature is between approximately 650° F. and approximately 750° F. The heat treatment anneals the material comprising the ribbon and provides it with a reliable shape for subsequent assembly steps. After heat-treatment, the braid retains its shape and its super-elastic properties.

Although the ribbons comprising the kink-resisting member 320 described above comprise a super-elastic alloy material, in other embodiments the ribbons comprise a braid made of a mixture of materials, such as a blend of super-elastic alloy and stainless steel components or of LCPs. Stainless steels and tungsten alloys can also be used. In certain embodiments, particularly in smaller diameter devices, more malleable metals and alloys, such as gold, platinum, palladium, rhodium, and so forth can be used. A platinum alloy with a few percent of tungsten has high opacity to radio frequency energy. Non-metallic ribbons and filaments can also be used; acceptable materials include, but are not limited to, high performance materials such as those made of polyaramids (for example, Kevlar®), LCPs and carbon fibers.

As used herein, the term “woven braid”, in addition to its ordinary meaning, further includes tubular constructions in which the ribbons, wires, or filaments comprising the construction are woven radially in an in-and-out fashion as they cross each other to form a tubular member having a single lumen. For example, the braid shown in FIG. 7B has a nominal pitch angle of 45°. Other braid angles from less than 10° to more than 60° can also be used. In other embodiments, the pitch angle of the braid is varied, either when it is woven or when it is included in the catheter section. In an exemplary embodiment, the innermost layer 322 has a smooth inner surface defining the delivery lumen. The delivery lumen and catheter axis 324 extends from the catheter distal end 106 to the catheter proximal end. In innermost layer 322 can comprise, for example, polymeric materials such as fluorocarbon polymers and lubricious polymers, including PTFE, ethylene-chlorofluoroethylene (“ECTFE”), fluorinated ethylene propylene (“FEP”), polychlorotrifluoroethylene (“PCTFE”), polyvinyl fluoride (“PVF”) or polyvinylidenefluoride (“PVDF”). Other materials such as polyethylene, polypropylene, polyvinylchloride (“PVC”), EVA, polyurethanes, polyamides, polyethyleneterephthalate (“PET”), polyamides (nylon) their mixtures, and co-polymers are also acceptable.

In certain embodiments wherein the innermost layer 322 comprises a fluorinated polymer, the outside surface of the innermost layer 322 can be etched to provide a good mechanical surface to which adjacent polymers will readily adhere. Certain procedures using, for example, treatment with a mixture of aliphatic hydrocarbons and sodium metal as the etching solution is effective in such service.

The kink-resisting member 320 can be placed directly adjacent innermost layer 322. In modified embodiments, kink-resisting member 320 is radially encased by one or more layers, such as an inner filler layer 326 and an outer filler layer 328. In such modified embodiments, the likelihood of slip or shift of the kink-resisting member 320 against the typically lubricious innermost layer 322 can be reduced. The filler layers 126, 128 adhere to the kink-resisting member 320 and form a determinate layer that enhances the kink-resisting capabilities of the catheter distal end 106. To soften the outer surface of the distal end 106 and to lower the stiffness of the distal end 106, a distal outer shaft layer 330 is placed on the outside surface of the filler layers 126, 128. In certain embodiments, the distal outer shaft layer 330 extends for substantially the entire length of the catheter distal end 106. The filler layers 126, 128 can be configured as extensions of tapered components of the assembly joint found in the junction section 306, discussed in greater detail below. Distal most sections made in this way can undergo bends of 1/32 inch diameter without visible kinking.

In an exemplary embodiment, the filler layers 326, 328 are similar materials. In one embodiment, the filler layers 326, 328 have a Shore hardness of approximately 45 D to approximately 60 D. In another embodiment, the filler layers 326, 328 have a Shore hardness of approximately 55 D. In one embodiment, the distal outer shaft layer 330 is a second material having a Shore hardness of between approximately 70 A and approximately 85 A. In another embodiment, the distal outer shaft layer 330 has a Shore hardness of approximately 75 A. The filler layers 326, 328 and the distal outer shaft layer 330 can comprise a variety of materials. In one embodiment, the filler layers 326, 328 and the distal outer shaft layer 330 comprise polymeric and selected other materials that tend to tack to each other upon heating. Such materials can also be melt-miscible. In other embodiments, the filler layers 326, 328 and the distal outer shaft layer 330 contain ancillary components that act as adhesives. The materials comprising the filler layers 326, 328 and the distal outer shaft layer 330 can be made of heat-shrinkable materials (for example, irradiated low-density polyethylene), or such materials can be otherwise placed onto the structure of the filler layers 326, 328 and the distal outer shaft layer 330. Examples of such materials include polyurethanes and their alloys, mixtures, and co-polymers. In certain embodiments, the filler layers 326, 328 and the distal outer shaft layer 330 comprise polymeric materials such as polyethylene, polypropylene, PVC, EVA, polyurethanes, polyamides, PET, and their mixtures and co-polymers. In other embodiments, the filler layers 326, 328 and the distal outer shaft layer 330 comprise mixtures of polyurethanes and polycarbonates sold as “Carbothane”.

As described above, the junction section 306 is located proximal to the distal end 106. The junction region 306 includes the region proximal to the distal end 106 that contains any tubing joint which has a tapering surface. FIG. 6 illustrates a modified embodiment in which several tapering surfaces are laminated together to form a long junction region 306. In an exemplary embodiment, the ratio of the length of the junction region to the diameter of the junction region is between approximately 12:1 and approximately 3:1. In another embodiment, the ratio of the length of the junction region to the diameter of the junction region is between approximately 5:1 and approximately 2.5:1.

In an exemplary embodiment, and as illustrated in FIG. 6, a first conical layer 332 having a proximal male conical surface is positioned adjacent the lubricious innermost layer 322. The first conical layer 322 can be an extension of inner filler layer 326. Likewise, a second conical layer 334 having a distal female conical surface that corresponds to the male surface on first conical layer 332. In certain embodiments, second conical layer 334 further comprises a distal male surface that corresponds to a proximal female conical surface on a third conical layer 336. In such embodiments, the third conical layer 336 is a proximal extension of the outer filler layer 328. Likewise, the second conical layer 334 is a distal extension of outer proximal layer 338. The outer proximal layer 338 comprises a material similar to the material found in the polymeric layers in catheter the distal section, such as the filler layers 326, 326 and the distal outer shaft layer 330. In certain embodiments, the outer proximal layer 338 comprises a material having a Shore hardness of between approximately 65 D and approximately 85 D. In one embodiment, the outer proximal layer 338 comprises a material having a Shore hardness of between approximately 70 D and approximately 75 D.

FIG. 8 is an exploded view of the components comprising an exemplary embodiment of the catheter distal end 106 and junction section 306. As illustrated in FIG. 8, the lubricious innermost layer 322 is at least partially surrounded by inner filler layer 326, which includes the first conical layer 332. The kink-resisting member 320 is positioned on the exterior surface of inner filler layer 326. Outer filler layer 328 includes, in certain embodiments, third conical layer 336, which includes an internal female conical surface. As illustrated, outer filler layer 328 is positioned exterior to kink-resisting member 320. The distal outer shaft layer 330 is positioned still further exterior to the outer filler layer 328, and is approximately the same length as kink-resisting member 320. The outer proximal layer 338, which includes a distally extending second conical layer 334, is illustrated with both a distal male conical surface and an inner female conical surface. In embodiments wherein the filler layers 326, 328 are extensions of the tapered components of the assembly joint, the filler layers 326, 328 will also be the distal ends of the conical members.

In certain embodiments, multiple polymeric layers are included in the junction section 306 and the catheter distal end 106. In other embodiments, at least one of the exterior and interior surfaces of the catheter are coated with a lubricious layer that is either chemically bonded to the surface or physically coated to the relevant catheter exterior surface. Exemplary procedures for producing bonded lubricious coatings are described U.S. Pat. Nos. 5,531,715 and 5,538,512.

The polymers noted herein can be filled with radiopaque materials such as barium sulfate, bismuth trioxide, bismuth carbonate, powdered tungsten, powdered tantalum and so forth. In such embodiments, the location of the various portions of the catheter can be radiographically visualized in the human body.

In other embodiments, the pitch of kink-resisting member 320 varies within the catheter distal end 106. In one such embodiment, the pitch of kink-resisting member 320 is greater towards the catheter distal end 106, thereby providing enhanced flexibility in that region.

The components described herein that have tapering surfaces can be manufactured by placing an appropriately sized tubing section on a mandrel having the sought shape. The tubing section is then stretched until the sought shape is achieved. The tubing section is then removed from the mandrel and is cut to the appropriate size.

In an exemplary catheter assembly technique, the lubricious innermost layer 322 is placed on a mandrel and axially stretched to produce axial molecular orientation. In an exemplary embodiment, the mandrel chosen provides an appropriate change in the innermost layer 322 inner diameter, as described above. The catheter elements are then assembled as illustrated in FIG. 8. A heat shrinkable tubing is then placed on the exterior of the catheter assembly and shrunk down to maintain the catheter elements in position and retain their position as they are further heated to cause the various polymers to flow into each other and form the conical surfaces as shown in FIG. 8. In some embodiments, the conical surfaces will not have the straight line interfaces illustrated in FIG. 8. In such embodiments, a significant amount of curvature may exist within the junction region.

Techniques for producing variable flexibility.

As described above, providing a ultrasound catheter with a variable flexibility can enhance maneuverability of the catheter through small vessels of a patient's vasculature. In particular, in certain embodiments a proximal region of the catheter has decreased flexibility to enhance pushability, torqueability and kink-resistance, while a distal region of the catheter has increased flexibility to allow the catheter to easily track a guidewire and to navigate small-radius bends of a patient's vasculature. Often, the distal end of an ultrasound catheter will have decreased flexibility in the region of the ultrasound radiating member.

FIG. 9 illustrates a catheter 100 having a variable flexibility. The catheter 100 has a small diameter distal end 106, a relatively larger diameter proximal end 104, and a delivery lumen 112 extending from the distal end 106 to the proximal end 104 of the catheter 100. The catheter 100 includes one of the stiffening mechanisms illustrated in FIGS. 10 through 20, which varies the stiffness of the catheter 100 along the length of the catheter 100, while introducing few, if any, discontinuous changes in flexibility of the catheter 100. In certain embodiments, the catheter 100 can be fitted with an ultrasound radiating member at its distal end, as illustrated in FIGS. 2A and 2B, but omitted in FIGS. 9 through 23 for clarity.

In the exemplary embodiment illustrated in FIGS. 9 through 12, the outer diameter of the catheter 100 increases gradually from the distal end 106 to the proximal end 104, while the delivery lumen 112 has a substantially constant diameter x. However, in certain embodiments the delivery lumen 112 is enlarged near the proximal end 104 to facilitate loading of the guidewire. The delivery lumen diameter x is between approximately 0.010 inches and approximately 0.020 inches, thereby allowing it to accommodate and closely fit standard and nonstandard sized guidewires. In such embodiments, the outer diameter of the catheter 100 is just slightly larger than the delivery lumen diameter x, from between approximately 0.025 inches and approximately 0.032 inches at the distal end 106, and gradually increasing to between approximately 0.030 inches and approximately 0.040 inches at the proximal end. In one embodiment, the outer diameter of the catheter 100 at the proximal end 104 is approximately 0.035 inches. As described above, the length of the catheter 100 can vary from less than approximately 60 cm to more than approximately 175 cm, depending on the application.

The exemplary embodiment illustrated in FIGS. 9 through 12 comprises one or more stiffener strands 150. The distal cross section corresponding to line d-d of FIG. 9 is shown in FIG. 10. As shown in FIG. 10, the outer sheath 108 comprises most of the overall catheter cross section at the distal end 106. At the catheter distal end 106, the stiffener strands 150 are small relative to the thickness of the outer sheath 108. In certain embodiments, the stiffener strands 150 taper away completely and disappear proximal to the ultrasound radiating member (not shown). For example, in one embodiment, the stiffener strands 150 end at a point approximately 30 cm proximal to the ultrasound radiating member.

The diameter of the delivery lumen 112, x, can vary depending on several factors, including the size of the guidewire to be used with the catheter. In FIG. 11, which shows the radial cross section of a midsection of the catheter along line m-m, the outer diameter of the outer sheath 108 is slightly larger than that at the catheter distal end 106. The stiffener strands 150 at the midsection are also slightly larger than at the catheter distal end 106. In FIG. 12, which shows the radial cross section of the proximal end of the catheter along line p-p, the outer diameter of the outer sheath 108 is still larger than at the midsection of the catheter, and the stiffener strands 150 are also larger. In certain embodiments, the stiffener strands 150 are sufficiently large such that they fuse together to form an intramural ring within the outer sheath 108.

In an exemplary embodiment, both the tubular body 108 and the stiffener strands 150 comprise polymers, including thermoplastics such as LDPE, HDPE, polypropylene, polystyrene, polyurethanes, polyesters (including nylon), polyfluorocarbons, and polyolefin. In other embodiments, the tubular body 108 and the stiffener strands 150 comprise composite materials, blends, and copolymers of the aforementioned compounds. For example, in one embodiment, the stiffener strands 150 comprise a material having a stiffness greater that the stiffness of the outer sheath 108. In such embodiments, the two materials can be miscible, such that the stiffener strands 150 will melt into the outer sheath 108 when extruded, and will form a catheter body without distinct boundaries between the stiffener strands 150 and the outer sheath 108.

In one exemplary embodiment, the catheter comprises an outer sheath 108 made of LDPE and polyolefin (ethylene octane) in approximately equal portions, and stiffener strands 150 made of a higher stiffness material, such as HDPE. The materials comprising the catheter can vary according to the intended use, and many other plastics and composite materials, and even metals, can be used. For example, in one embodiment, the outer sheath 108 comprises LDPE, and the stiffener strands 150 comprise HDPE, LDPE, or a mixture of the two.

In a modified embodiment, the relative stiffness of the materials comprising the outer sheath 108 and the stiffener strands 150 is reversed, with the outer sheath 108 comprising the stiffer material, and the stiffener strands 150 comprising the more flexible material. In such embodiments, the stiffener strands 150 will be thicker at the catheter distal end 106 and thinner at the catheter proximal end 104, thereby providing the catheter with increasing flexibility distally.

FIGS. 13 through 15 illustrate the distal, midsection, and proximal cross sections of a catheter having variable diameter stiffener strands 150 and a substantially constant outer diameter. The stiffener strands 150 increase in thickness from the distal cross section d-d of FIG. 13 to the proximal cross section p-p of FIG. 15, but the outer diameter of the outer sheath 108 remains substantially constant along the length of the catheter. The inner diameter x of the delivery lumen 112 also remains substantially constant along the length of the catheter. Accordingly, as the quantity of stiffener strand material decreases from the catheter proximal end 104 to the catheter distal end 106, the quantity of outer sheath material increases by approximately the same quantity.

FIGS. 16 through 18 illustrate the distal, midsection, and proximal cross sections of a catheter having a variable thickness inner stiffener layer. In such embodiments, rather than using stiffener strands of increasing thickness proximally, an inner stiffener layer 152 having a gradually increasing thickness proximally is used. The outer sheath 108 is more flexible than the inner stiffener layer 152. In one embodiment, the outer layer 108 comprises, for example, LDPE or a mix of LDPT and polyolefin, and the inner stiffener layer 152 comprises, for example, HDPE. A comparison of the thicknesses of the inner stiffener layers 152 illustrated in FIGS. 16 through 18 reveals that the inner stiffener layer 152 becomes gradually thicker and comprises a larger portion of the catheter wall from the distal cross section d-d to the proximal cross section p-p. FIG. 19 illustrates the longitudinal cross section of a catheter 100 corresponding to the three radial cross sections of FIGS. 16 through 18. As illustrated, the thickness of inner stiffener layer 152 gradually increases from the catheter distal end 106 to the catheter proximal end 104. As described above, such a catheter can have a substantially uniform outer diameter along its length, or it can have an outer diameter that increases toward the catheter proximal end. In certain embodiments, the catheter distal end 106 can include a segment adjacent the ultrasound radiating member (not shown) where the inner stiffener layer 152 is absent.

FIG. 20 illustrates a catheter having a non-discretely gradually increasing stiffness proximally. Specifically, no discrete stiffener strands are present in this embodiment. Instead, the composition of the outer sheath 108 gradually changes along the length of the catheter from a first material to a second material. For example, in the catheter distal end 106, the outer sheath 108 comprises a more flexible material, for example LDPE, and in the catheter proximal end 104, the outer sheath 108 comprises a less flexible material, such as HDPE. Between the distal and proximal ends, the composition of the outer sheath 108 gradually changes from one material to another (for example, from predominantly LDPE to predominantly HDPE). The stippling shown in the cross section of FIG. 12 indicates the gradual transition of the catheter wall from LDPE to HDPE.

In modified embodiments, the catheters described herein can further include one or more radiopaque markers to assist in positioning the catheter in, and navigating the catheter through, a patient's vasculature.

The catheters described herein can be used in the highly tortuous blood vessels of the body, including the coronary blood vessels, renal blood vessels, and intracranial blood vessels. As used herein, the term “highly tortuous” refers, in addition to its ordinary meaning, to the tortuosity typically encountered in the vascular pathway from a remote access site such as the femoral artery to target sites deep within the coronary, renal sinus and cerebral vasculature. Specific catheter embodiments can be constructed for access into targeted sites involving pathologically tortuous blood vessels. As used herein, the term “pathological tortuosity” refers, in addition to its ordinary meaning, to the vascular pathway from a remote access site such as the femoral artery to target sites involving (a) turns in excess of 90°, such as encountered when branching from one blood vessel to another blood vessel (that is, paths that branch off the preceding vessel at angles greater that a right angle), and (b) a total path length within the target tissue at least approximately 5 cm. Pathological tortuosity includes treatment sites accessible by a guidewire approximately 0.018 inches or smaller.

The variable flexibility catheters described herein can be used with a guidewire, although a guidewire is not required. The catheter flexibility can be varied to allow the catheter to be guided to the treatment site in a flow directed manner, or through manual steering. The materials and dimensions described herein can be varied so that the catheter can be used in highly tortuous pathways with or without a guidewire. The variable flexibility catheters described herein can increase catheter maneuverability despite the presence of a relatively rigid ultrasound radiating member at the distal end of the catheter.

The variable flexibility catheters described herein can be manufactured using various known extrusion methods. Known methods of co-extrusion, including for example cross header arrangements, over-extrusion, and extrusion die construction can be applied to manufacture these catheters. Stiffener strand thickness, wall thickness, and relative percentage of outer sheath composition can be controlled with known techniques including for example speed controlled extrusion, throttled flow controlled extrusion, and waste-gating. The materials disclosed herein can be used in catheter fabrication, but it is expected that new and improved materials will also be applied in the construction of the catheters disclosed herein.

For example, in one exemplary method of manufacture, a catheter is manufactured by an extrusion method in which the tubular catheter having a plurality of stiffener strands is co-extruded from a first material forming the outer sheath 108, and a second material forming the stiffener strands 150. The diameter of the stiffener strands 150 can be varied during the extrusion process to form a catheter having a changing flexibility along its length.

In another exemplary method of manufacture, the catheter is extruded as a tubular member having a substantially constant cross-sectional configuration that includes a first outer sheath material and a second stiffener strand material. The extruded tubular member having a substantially constant cross-sectional configuration is then heated and stretched to a final configuration in which the catheter distal end is smaller in diameter and more flexible than the catheter proximal end. The cross sections of a distal, intermediate, and proximal portion of a catheter formed by this method are illustrated in FIGS. 21 through 23, respectively. As illustrated, as the catheter is stretched, the stiffener strands 150 decrease in diameter and move closer together. The overall dimensions of the catheter decrease distally. In one embodiment, the stiffener strands 150 contact each another and melt together into an inner stiffener layer at the catheter distal end as the catheter is stretched.

Multi-Segment Catheter.

As described above, providing a ultrasound catheter with a variable flexibility can enhance maneuverability of the catheter through small vessels of a patient's vasculature. In particular, in certain embodiments a proximal region of the catheter has decreased flexibility to enhance pushability, torqueability and kink-resistance, while a distal region of the catheter has increased flexibility to allow the catheter to easily track a guidewire and to navigate small-radius bends of a patient's vasculature. Often, the distal end of an ultrasound catheter will have decreased flexibility in the region of the ultrasound radiating member.

FIG. 24 illustrates an intermediate portion of catheter having a variable stiffness along its axial length. Such a catheter can be used to deliver an ultrasound radiating member to a treatment site within a patient's vasculature. In the exemplary embodiment illustrated in FIG. 24, the catheter comprises an outer tube 918 and three coaxial inner tubular segments 919, 920, 921. As illustrated, the three coaxial inner tubular segments 919, 920, 921 are disposed in tandem within the outer tube 918, and are contiguous to each other.

The outer tube 918 extends over substantially the entire length of the catheter, which can be over approximately 50 cm, and is between approximately 80 cm and approximately 150 cm in certain embodiments. (As described above, in certain embodiments the outer tube 918 does not cover an ultrasound radiating member positioned at the catheter distal end.) The outer diameter of outer tube 918 (as measured at the catheter proximal end) can be between approximately 0.75 mm and 2.00 mm, and is between approximately 0.85 mm and 1.30 mm in certain embodiments. In a modified embodiment, the outer tube 918 necks down at its distal end, such that its outer diameter at the distal end is slightly smaller than at its proximal end. The outer tube 918 can have a wall thickness of between approximately 0.08 mm and approximately 0.16 mm, and has a wall thickness of between approximately 0.10 mm and approximately 0.13 mm in certain embodiments. In an exemplary embodiment, the outer tube 918 comprises a polymer having a flexural modulus (as measured by ASTM D-790) of between approximately 100,000 kPa and approximately 250,000 kPa, such as low density polyethylene.

In the exemplary embodiment illustrated in FIG. 24, the proximal inner tubular segment 919 extends from the catheter proximal end to proximal junction 922. In one embodiment this distance is between approximately 10 cm and approximately 70 cm, in another embodiment this distance is between approximately 40 cm and approximately 60 cm, and in yet another embodiment this distance is approximately 50 cm. In one embodiment the wall thickness of the proximal inner tubular segment 919 is between approximately 0.08 mm and approximately 0.18 mm, and in another embodiment the wall thickness of the proximal inner tubular segment 919 is between approximately 0.10 and approximately 0.13 mm. In one embodiment, the proximal inner tubular segment 919 comprises a polymer having a flexural modulus of between approximately 1,500,000 kPa and approximately 1,800,000 kPa, such as polypropylene. Thus, in such embodiments, the portion of the catheter between the catheter proximal end and the proximal junction 922 is relatively stiff. In one embodiment, the inner diameter of the proximal inner tubular segment 919 is between approximately 0.45 mm and approximately 0.75 mm.

Referring still to the exemplary embodiment illustrated in FIG. 24, intermediate inner tubular segment 920 extends from proximal junction 922 to intermediate junction 923. In one embodiment the length of the intermediate inner tubular segment 920 is between approximately 30 cm and approximately 100 cm, in another embodiment the length of the intermediate inner tubular segment 920 is between approximately 70 cm and approximately 90 cm, and in yet another embodiment the length of the intermediate inner tubular segment 920 is approximately 80 cm. In an exemplary embodiment, the intermediate inner tubular segment 920 is less stiff than proximal inner tubular segment 919. Accordingly, the wall thickness of the intermediate inner tubular segment 920 is less than the wall thickness of proximal inner tubular segment 919. For example, the intermediate inner tubular segment 920 can comprise a polymer having a lower flexural modulus than the polymer comprising the proximal inner tubular segment 919. In a modified embodiment, the intermediate inner tubular segment 920 comprises the same polymer as the proximal inner tubular segment 919, but has a smaller wall thickness. In one embodiment the intermediate inner tubular segment 920 has a wall thickness of between approximately 0.05 mm and approximately 0.13 mm, and in another embodiment the intermediate inner tubular segment 920 has a wall thickness of between approximately 0.05 mm and approximately 0.08 mm. In an exemplary embodiment, the inner tubular segments 919, 920 can comprise a continuous length of tubing having an appropriately tapered outer diameter.

The distal inner tubular segment 921 extends from intermediate junction 923 to a location 924 proximal to the distal end of the catheter. For example, in one embodiment, location 924 can be adjacent the proximal end of an ultrasound radiating member. In one embodiment the length of the distal inner tubular segment 921 is between approximately 5 cm and approximately 20 cm, in another embodiment the length of the distal inner tubular segment 921 is between approximately 7 cm and approximately 15 cm, and in yet another embodiment, the length of the distal inner tubular segment 921 is approximately 10 cm.

Based on the foregoing, in an exemplary embodiment, the distance from proximal junction 922 to the distal end of the catheter will be greater than approximately 50% of the entire length of catheter. In another embodiment, the distance from proximal junction 922 to the distal end of the catheter will be greater than approximately 60% of the entire catheter length. In such embodiments, distal inner tubular segment 921 is less stiff than intermediate inner tubular segment 920, and provides a transition in flexibility between inner tubular segment 920 and the portion of the outer tube 918 that extends distal to location 924. In such embodiments, the wall thickness of distal inner tubular segment 921 (a) is less than that of intermediate inner tubular segment 920, and/or (b) comprises a polymer having a lower flexural modulus than the polymer comprising intermediate inner tubular segment 920. For example, in one embodiment, distal inner tubular segment 921 comprises a polymer having a flexural modulus that is (a) significantly lower than the polymer comprising intermediate inner tubular segment 920 but (b) higher than the polymer comprising the outer tube 918. The distal inner tubular segment 921, for instance, can be linear, low density polyethylene. Typically, the flexural modulus of the polymer comprising distal inner tubular segment 921 is between approximately 150,000 kPa and approximately 350,000 kPa. In one embodiment, , the flexural modulus of the polymer comprising distal inner tubular segment 921 is between approximately 200,000 kPa and approximately 300,000 kPa. In one embodiment the wall thickness of distal inner tubular segment 921 is between approximately 0.05 mm and 0.10 mm, and in another embodiment the wall thickness of distal inner tubular segment 921 is between approximately 0.06 mm and 0.09 mm. In an exemplary embodiment, the inner diameters of segments 920, 921 are substantially the same as that of segment 919.

Although junctions 922, 923 are illustrated as butt joints in FIG. 24, these junctions can comprise other types of joints in other embodiments. For example, in one other embodiment, junctions 922, 923 comprise overlap joints.

Thus, the catheter illustrated in FIG. 24 comprises four segments of different flexibility or stiffness, with the segments becoming increasingly flexible distally. This configuration provides a more gradual flexibility or stiffness gradient than a two-segment catheter. Specifically, the change in flexibility or stiffness between catheter segments in the embodiments described herein is not as great in a two-segment catheter. In particular, the catheter embodiments described herein allow the distal end of the catheter to be tracked around sharp bends in a patient's vasculature with less likelihood of kinking, despite the presence of a rigid ultrasound radiating member located at the distal end of the catheter. The multi-segment configuration improves the ability of the catheter to track a guidewire around sharp bends, and reduces the likelihood of fatigue stress failure, delamination, or other catheter structural failure. Such multi-segment catheters can be manufactured according to the catheter manufacturing techniques disclosed herein.

Proximal element joint.

Several techniques for varying the flexibility, stiffness and other mechanical properties of a catheter body are disclosed herein. As described elsewhere in this specification, in certain embodiments the catheter body is less flexible at the catheter proximal end, and gradually increases in flexibility toward the distal end. This configuration advantageously enhances catheter maneuverability by facilitating the pushing, twisting or other motions used when advancing the catheter over a guidewire and through a patient's vasculature to a treatment site. For example, a catheter with increasing distal flexibility often has enhanced kink resistance.

However, as described above, many of the techniques for manipulating the mechanical properties of the catheters described herein can be used with a catheter having one or more ultrasound radiating members mounted in a catheter distal region. In such embodiments, the ultrasound radiating member acts as a relatively stiff tip in the end region of an otherwise flexible catheter. Thus, at the proximal element joint there is a discontinuous change in flexibility from the relatively flexibility distal region of the outer sheath to the relatively rigid ultrasound radiating member. As described previously, reducing the rigidity of the proximal element joint enhances the joint flexibility, reduces the likelihood of kinking in the catheter flexible support section, and facilitates tracking of the catheter over the guidewire.

Thus, in an exemplary embodiment, any of the techniques described herein from introducing a gradually increasing flexibility from the catheter proximal region to the catheter distal region can also be used to introduce a variable flexibility at the proximal element joint. For example, the flexibility of the outer sheath can gradually decrease in a region proximal to the ultrasound radiating member, thereby eliminating the discontinuous change in flexibility at the proximal element joint.

The relative catheter flexibility as a function of axial catheter position of such an exemplary embodiment is illustrated in FIG. 25. In particular, FIG. 25 illustrates the relative flexibility of an ultrasound catheter (on the y-axis) at various points along the catheter length (x-axis) between the catheter proximal region and the distal tip. As described above, in regions where the catheter is becoming more flexible distally, denoted as increasing flexibility region 160 in FIG. 25, any of the methods described herein for providing variable catheter flexibility can be used.

Likewise, in the proximal element joint region 162, where the catheter is becoming less flexible distally, any of the methods described herein for providing variable catheter flexibility can be used. Such methods include, but are not limited to, use of braids, compression regions, stiffener wires, and composite materials. Such methods can be employed to eliminate a discontinuous change in catheter flexibility at the proximal element joint between the catheter distal region and the relatively rigid ultrasound radiating member region 164.

Still referring to FIG. 25, the distal tip region 166, which is located distal to the ultrasound radiating member region 164, can be provided with an increasing flexibility to enhance maneuverability through the patient's vasculature.

FIG. 25 represents an exemplary configuration of a variable flexibility catheter. Other configurations can be used in other embodiments. For example, in a modified embodiment, the catheter can have a relatively constant flexibility between the proximal region and the distal region, with a variable flexibility at the proximal element joint. Such an embodiment can comprise fewer components, and thus have a reduced manufacturing cost. In still other embodiments, the catheter can have a plurality of flexibility maxima and minima between the catheter proximal region and the catheter distal region, with such maxima and minima positioned axially depending on the characteristics of the vasculature through which the catheter is to be routed.

With continued reference to FIG. 25, in one embodiment the ultrasound radiating member region 164 comprises a portion that of the catheter that is substantially unbendable during normal use conditions. In one embodiment, the length of the ultrasound radiating member region 164 has a length that is less than about 6 mm, in another embodiment, less than about 5 mm and, in yet another embodiment, less than about 4 mm. In these embodiments, the ultrasound radiating member region 164 has a length greater than about 3 mm such that sufficient energy (i.e., ultrasound energy in the preferred embodiment) can be delivered to the treatment site. Catheters with substantially unbendable energy delivery sections of longer lengths have difficulty effectively navigating small blood vessels, such as the main and subsequent branches of the middle cerebral artery.

Delivery lumen with composite tubing.

As described above, if the ultrasound catheter buckles or kinks during advancement through the patient's vasculature, it may not be possible to deliver the ultrasound radiating member to the treatment site. With respect to the neurovascular system, this is an important technical hurdle that has limited the use of ultrasound catheters. Furthermore, buckling or kinking of the catheter can damage the patient's vasculature.

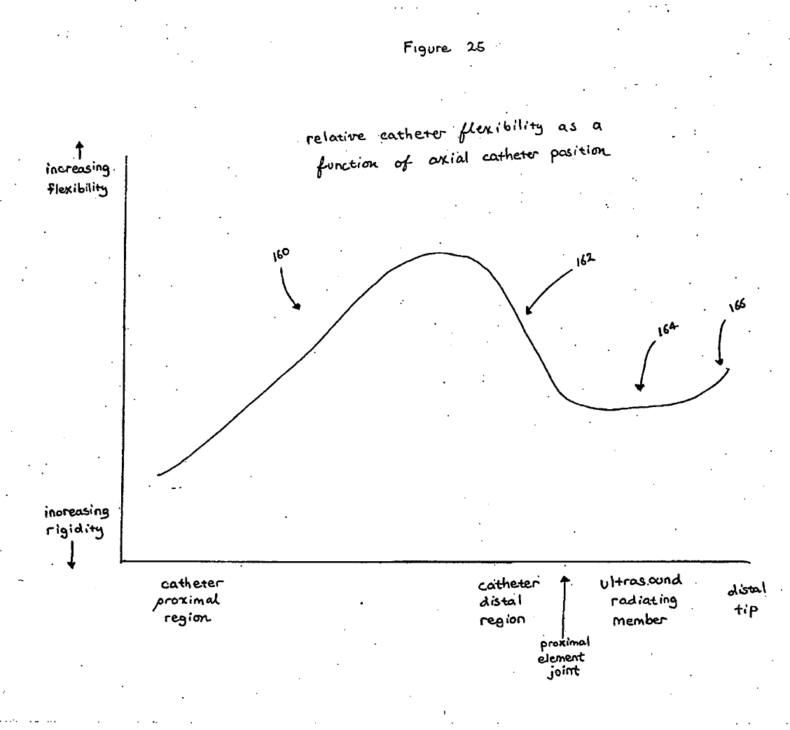

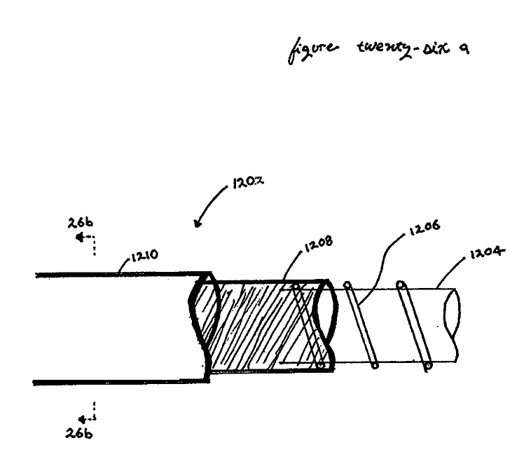

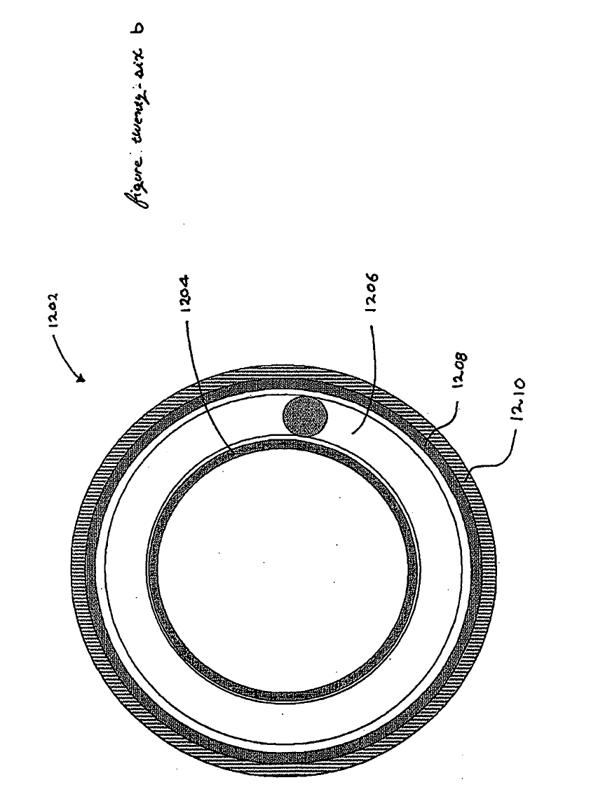

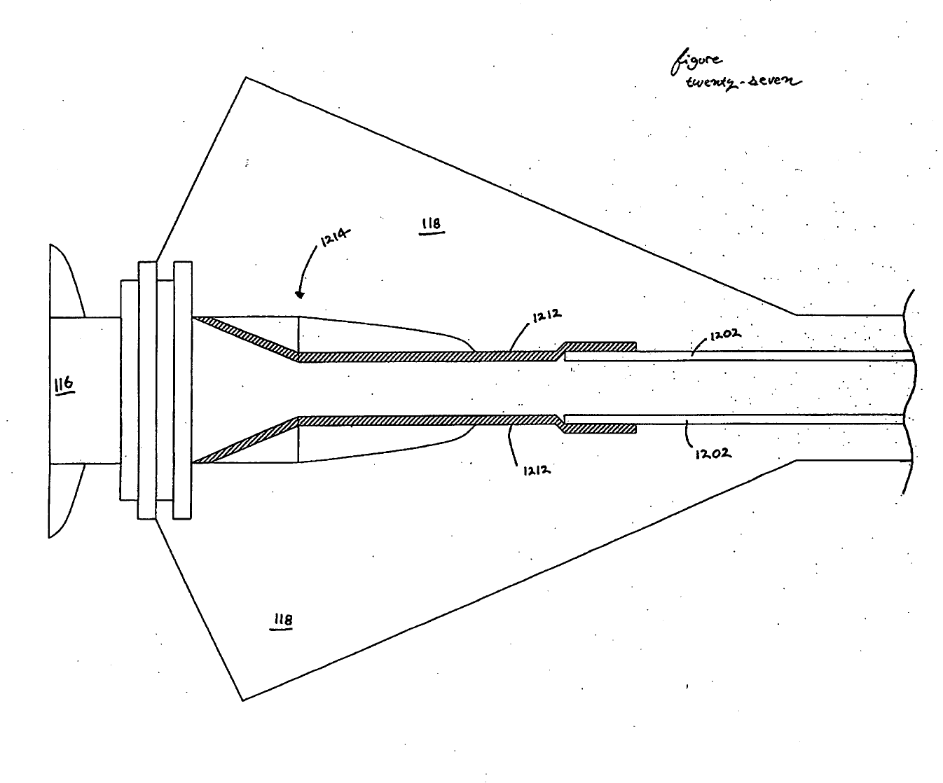

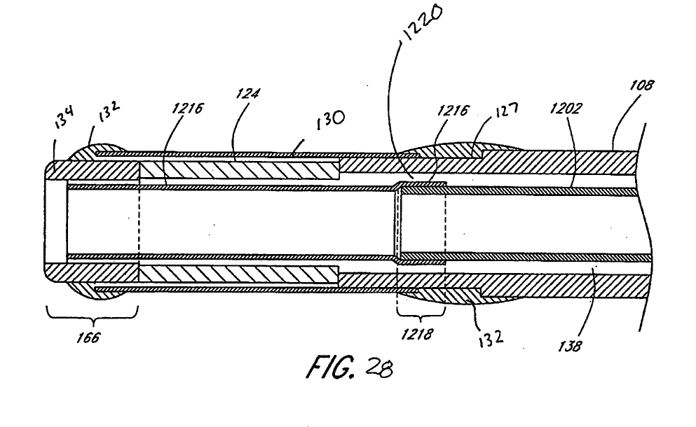

With respect to the ultrasound catheter described above, it is particularly advantageous that the inner core 110 does not undergo kinking or distortion (also referred to as “ovalization”) when the catheter is passed through difficult regions of the patient's vasculature. Such ovalization will cause the inner core 110 to bind on the guidewire over which the catheter is advanced. Thus, FIGS. 26A and 26B illustrate an improved inner core 1202, which is configured to provide enhanced resistance to kinking and buckling, while retaining sufficient flexibility to enable navigation through difficult regions of the patient's vasculature.

As shown in FIGS. 26A and 26B, the inner core 1202 has a composite construction that comprises an inner member 1204 surrounded by a reinforcing member 1206, which is, in turn, is preferably overlaid with an outer member 1209. In the illustrated embodiment, the inner member 1204 is preferably made of a lubricious material, such as, for example, Teflon®. The reinforcing member 1206 preferably comprises a stainless steel wire, which is coiled around the inner member 1204 in an helical pattern having a density of about 40 wraps per inch. In modified embodiments, the inner member 1204 may be arranged about the inner member 1204 in a different pattern (e.g., woven, zig-zag etc.) and may be formed of a different material (e.g., gold, other metals or alloys, fibers, etc.). In a preferred embodiment, the coil comprises a wire with a flattened profile but in other embodiments, the coil may have other cross-sectional shapes (e.g., round). In still another embodiment, the member 1206 may be formed of a hypotube or a polymer tube in which cuts (e.g., helical cuts) are formed. In general, the reinforcing member 1206 provides radial strength while being capable of at least limited longitudinal expansion and contraction. The limited longitudinal expansion and contraction provides the inner core 1202 with sufficient flexibility. For example, the reinforcing member 1206 may expand and contract as the catheter bends.

In the illustrated embodiment, the outer member 1209 preferably comprises a lubricious polymer that can be coated with a layer of an additional material . In the preferred embodiment, the outer member comprise a Pebax® wall 1208 that is coated with a Tecoflex® outer skin 1210. Of course, other materials can be used in other embodiments.

In one embodiment, the inner member 1204 comprises a 0.005 inch thick Teflon layer. The reinforcing member 1206 comprises a 0.0005 inch thick flat wire stainless steel wire coil, wrapped around the Teflon layer. A Pebax layer is overlayed over the wire coil and Teflon layer. The Pebax layer has a thickness of about 0.00075 inches over the wire and about 0.00125 inches over the gaps in the wire. A layer of Tecoflex having a thickness of about 0.00025 is preferably provided over the Pebax layer.

As mentioned above, in the preferred embodiment, the inner core 1202 is configured such that at least a portion of the reinforcing member 1206 can flex with respect to the longitudinal axis of the catheter. In between freely flexing portions, the reinforcing member 1206 may be fixed with respect to the inner member 1204 and/or outer member 1209. In one preferred embodiment, the reinforcing member 1206 may be fixed with respect to the inner member 1204 and/or outer member 1209 only at the distal and proximal portions or ends of the inner core 1202.

In one embodiment, the reinforcing member 1206 extends over at least about 50% of the length of the catheter. In another embodiment, the reinforcing member 1206 extends over at least about 75% of the length of the catheter. In another embodiment, the reinforcing member 1206 extends over substantially the entire length of the catheter. These arrangements advantageously allow the reinforcing member to flex as the catheter is advanced through torturous anatomy, while still reducing ovalization.

As mentioned above, guidewire movement can be hindered by kinking or distortion (also referred to as “ovalization”) of the catheter body. Kink resistance of the ultrasound catheter, which is also related to the ability to freely pass a guidewire through the catheter, can be evaluated by testing the minimum radius 180° bend that the catheter can be subjected to without kinking. In an exemplary embodiment, the catheter with the composite inner core 1202 described above can be subjected to a 180° bend having a radius of less than about 10 mm without kinking. In another exemplary embodiment, the catheter can be subjected to a about 180° bend having a radius of less than about 8 mm without kinking. In still another exemplary embodiment, the catheter can be subject to a 180° bend having a radius of less than or equal to about 6 mm without kinking. In such embodiments, the inner core 1202 is configured to receive a standard 0.014 inch guidewire, which may be up to about 0.017 inches in diameter. In one embodiment, the inner diameter of the inner member 1204 is approximately 0.018 inches±0.005 inches. In other embodiments, the inner diameter of the internal liner 1204 is approximately 0.018 inches±0.010 inches. In still other embodiments, the inner diameter of the internal liner 1204 is approximately 0.018 inches±0.100 inches.

In embodiments wherein the ultrasound catheter includes the composite delivery lumen 1202 described herein, the kink resistance and flexibility of the catheter 100 is advantageously increased, as compared to a catheter with a delivery lumen consisting solely of polyimide. This configuration also reduces the tendency of the tubular body to become ovular when passed through difficult regions of the patient's vasculature, thereby reducing the likelihood of binding of the guidewire within the delivery lumen 1202. Additionally, the presence of the reinforcing member 1206 increases the burst strength, kink resistance and flexibility of the delivery lumen 1202, and provides for a stronger bond at locations where other catheter components are to be bonded to the delivery lumen 1202—such as at the distal and proximal ends of the delivery lumen 1202. It should also be appreciated that the reinforcing member 1206 may be used to control the flexibility of the delivery lumen 1202 and, in turn, the catheter. This may be done by varying the thickness and/or properties of the reinforcing member 1206.

In certain embodiments, the delivery lumen can be configured with dimensions to increase the size of the region 138 (see FIG. 2A) between the delivery lumen and the outer sheath. Providing a larger region 138 allows more room for electrical conductors—for example, conductors configured to provide power to the ultrasound radiating member(s) and temperature sensor(s)—to be positioned therein.

The techniques for increasing the maneuverability of the tubular body described herein can be applied to the entire length of the tubular body, or can be applied to a portion of the tubular body. In other embodiments, the techniques can be applied along different lengths of the catheter to varying degrees. For example, in one such embodiment, the tubular body can be configured with a varying flexibility, such that the flexibility of the tubular body gradually increases from the proximal region to the distal region as described above. Also as described above, other characteristics of the tubular body, such as kink resistance and torqueability, can be can be varied along the length of the catheter.

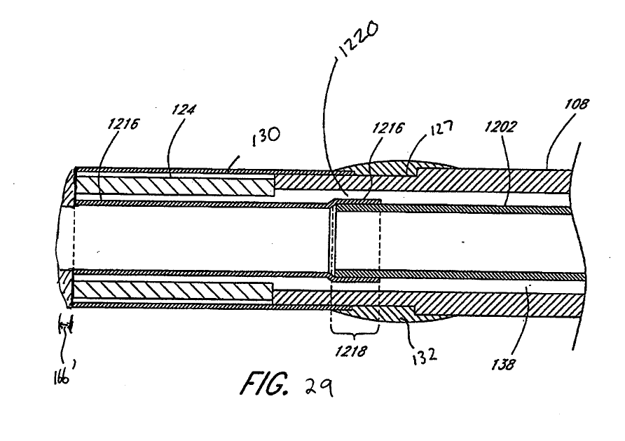

In embodiments wherein the delivery lumen comprises a composite delivery lumen 1202 as described above, and as illustrated in FIGS. 26A and 26B, a polyimide sleeve can be incorporated into the backend hub 118 (see FIG. 1) to facilitate mating of the composite delivery lumen 1202 with the backend hub 118. For example, FIG. 27 illustrates selected internal components of the backend hub 118 that can be used in connection with the composite delivery lumen 1202.

For example, the backend hub 118 illustrated in FIG. 27 includes a polyimide sleeve 1212 that is bonded to the proximal end of the composite delivery lumen 1202. In an exemplary embodiment, the polyimide sleeve 1212 has an inner diameter substantially equal to the inner diameter of the composite delivery lumen 1202. One end of the polyimide sleeve 1212 can be expanded over the composite delivery lumen 1202 to create a secure slip-fit joint with a relatively smooth transition along the inner diameter. Heat and/or adhesives can be used to bond and seal the joint. This configuration advantageously facilitates passage of a guidewire through the backend hub 118 and into the composite delivery lumen 1202. Additionally, this configuration advantageously reduces or prevents exposure of the composite delivery lumen 1202 to ultraviolet light during curing operations, and reduces the amount of bending stress that the polyimide sleeve—composite delivery lumen 1202 joint is subjected to during assembly. In one embodiment, the length of the joint between the polyimide sleeve 1212 and the composite delivery lumen 1202 is approximately equal to the length of the proximal element joint, as defined above.

The other end of the polyimide sleeve 1212 is engaged with a Luer fitting 1214 in the backend hub 118 to anchor the polyimide sleeve 1212 in place. In an exemplary embodiment, the length of engagement between the polyimide sleeve 1212 and the Luer fitting 1214 is approximately 0.400 inches, although other dimensions can be used in other embodiments.

In embodiments wherein the delivery lumen comprises a composite delivery lumen 1202, a polyimide tube 1216 can be bonded to the distal end of the composite delivery lumen 1202, as illustrated in FIG. 28. The polyimide tube 1216 serves as a delivery lumen through the region of the ultrasound radiating member 124. The bond between the distal end of the composite delivery lumen 1202 and the polyimide tube 1216, referred to herein as the “distal delivery lumen bond” 1218, is located within the outer sheath 108 in an exemplary embodiment. In an such embodiments, the distal delivery lumen bond 1218 has a length between approximately about 0.020 inches and about 0.025 inches. In another embodiment, the distal delivery lumen bond 1218 has a length between approximately 0.010 inches and 0.035 inches. Other dimensions can be used in other embodiments. For example, in one embodiment, the distal delivery lumen bond 1218 has the minimum length permissible while still providing sufficient strength to hold the composite delivery lumen 1202 and the polyimide tube 1216 together.

Still referring to the exemplary embodiment illustrated in FIG. 28, the polyimide tube 1216 passes through the inner diameter of the ultrasound radiating member 124. In one such embodiment, the polyimide tube 1216 has an inner diameter of approximately 0.023 inches at the distal delivery lumen bond (where it fits over the composite delivery lumen 1202), and has an inner diameter of approximately 0.018 inches within the ultrasound radiating member 124. In such embodiments, the length of the polyimide tube 1216, including the length of the distal delivery lumen bond 1218, is between about 0.151 inches and about 0.182 inches. Other dimensions for the polyimide tube 1216 can be used in other embodiments.

The configuration of the distal delivery lumen bond 1218 described herein advantageously provides a secure, slip-fit joint between the composite delivery lumen 1202 and the polyimide tube 1216. The distal delivery lumen bond 1218 has a relatively smooth transition along the inner diameter. Heat can be used to bond and seal the joint; no adhesive is necessary, although an adhesive can be used in a modified embodiment. Using heat to bond the joint advantageously provides a high bond strength, allows close control of any reflow of the delivery lumen inner diameter, and provides a relatively small, low profile bond. However, other bonding techniques can be used in other embodiments.

The distal delivery lumen bond 1218 configuration described herein advantageously facilitates passage of a guidewire through the distal delivery lumen bond 1218, and generally improves the flexibility of the proximal element joint, thereby enhancing catheter accessibility to the distal vasculature. This configuration also covers sharp ends which can be present at the distal end of the composite delivery lumen 1202, such as from the coil 1206.

Furthermore, the presence of the distal delivery lumen bond 1218 in region 138 between the composite delivery lumen 1202 and the outer sheath 108 creates a narrow passage 1220 which can be used to hold an electrical conductor (not shown) in place, such as the electrical conductors used to drive the ultrasound radiating member. This configuration can reduce the likelihood of accidental disconnection of the electrical conductor from the ultrasound radiating member.

In a modified embodiment, the catheter can be etched in the region of the distal delivery lumen bond 1218. The etching generally increases the strength of the distal delivery lumen bond 1218.

With continued reference to FIG. 28, in this embodiment, the exemplary the distal end 106 of the catheter 100 also includes a sleeve 130 that is generally positioned about the ultrasound radiating member 124. As described above, the sleeve 130 comprises a material that readily transmits ultrasonic energy. Suitable materials for the sleeve 130 include, but are not limited to, polyolefins, polyimides, polyesters and other materials that readily transmit ultrasonic energy with minimal absorption of the ultrasonic energy. The proximal end of the sleeve 130 can be attached to the outer sheath 108 with an adhesive 132. To improve the bonding of the adhesive 132 to the outer sheath 108 and to improve flexibility, a shoulder 127 or notch is formed in the outer sheath 108 for attachment of the adhesive 132 thereto. In one embodiment, the notch 127 is formed by grinding down the outer diameter of the outer sheath 108. As shown in FIG. 28, the distal delivery lumen bond 1218 is preferably located within the same axial region as the adhesive bond between the outer sheath 108 and the sleeve 130.

Potting material may be placed between the ultrasound radiating member 124 and the sleeve 130 and/or polyimide tube 1216. The potting material reduces movement between these members and provides electrical insulation.

As described above, in the embodiment illustrated in FIGS. 1 through 2B, ultrasonic energy is generated from electrical power supplied to the ultrasound radiating member 124. The electrical power can be supplied through conductive wires 126, 128 that extend through the tubular body 102 between the outer member 108 and the inner core 1202. In a preferred embodiment, the conductive wires 126, 128 can extend freely in the region 138 between the inner core 1202 and the outer sheath 108. In the illustrated embodiment, the first wire 126 is connected to the hollow center of the ultrasound radiating member 124, while the second wire 128 is connected to the outer periphery of the ultrasound radiating member 124.

As described with the embodiment of FIG. 2B, the at least one temperature sensor (not shown) is positioned on or near ultrasound radiating member 124. A control wire 127 connects the sensor to the control box. As with the conductive wires 126, 128, the control wire 127 can extend through the tubular body 102 between the outer member 108 and the inner core 1202 and preferably extend freely in the region 138 between the inner core 1202 and the outer sheath 108.

In a preferred embodiment, the portions of the conductive wires 126, 128 and/or the control wire 127 positioned between the outer and inner components of the catheter has an extended length that is longer than the extended length of the corresponding outer and inner components of the catheter. In this manner, the wires 126, 127, 128 have slack such that, as the catheter is advanced through the vascular system, the wires 126, 127, 128 do not substantially add to the stiffness of the catheter. In addition, because the wires 126, 127, 128 can freely move they can bend and compress further reducing the stiffness of the catheter. In one embodiment, the portions of the conductive wires 126, 128 and/or the control wire 127 positioned between the outer and inner components of the catheter has an extended length that is in the range of about 0.02% longer than the extended length of the corresponding outer and inner components of the catheter. In another embodiment, the extend length is at least about 0.5% longer, and in another embodiment at least about 0.70% longer. In one embodiment, the extend length is in the range of about 0.02% to about 0.70% longer than the extended length of the corresponding outer and inner components of the catheter and in another embodiment, the extended length is in the range of about 0.02% to about 0.50% longer. In these embodiments, the wires 126, 127, 128 may be wrapped (e.g., helically wrapped) about the inner core 1202 to take up the extra length.

Ultrasound catheter with reduced distal rigid section.

As described previously, the ultrasound catheter often has a region of decreased flexibility in the distal region of the catheter around the ultrasound radiating member (see, for example, ultrasound radiating member region 164 in FIG. 25). This distal rigid section can impede passage of the catheter through difficult regions of the patient's vasculature, especially as the length of the distal rigid section increases. This difficulty is often manifested when the flexible distal tip (see, for example, distal tip region 166 in FIG. 25) becomes ovular and pinches the guidewire during tracking of the ultrasound catheter over the guidewire.

The ability of the ultrasound catheter to reliably track the guidewire can be improved by decreasing the length of the distal tip region 166. For example, in one embodiment of an ultrasound catheter with improved guidewire tracking performance, the length of the distal tip region 166 is between approximately 0.35 inches and approximately 0.45 inches. Indeed, implementation of design improvements such as this allow the length of the ultrasound radiating member 124 to be increased—thereby advantageously allowing more ultrasonic energy to be delivered to the treatment site—without adversely affecting the ability of the ultrasound catheter to reliably track the guidewire in distal regions of the patient's vasculature. Furthermore, decreasing the length of the distal tip region 166 advantageously reduces the tendency of the distal exit port 114 to open and become folded back on itself (commonly referred to as “fishmouthing”) as the catheter is passed through the patient's vasculature.

FIG. 29 illustrates another embodiment in which the length distal tip region 166′ has been reduced significantly to form a blunt, atraumatic tip. The distal tip region 166′ is preferably relatively hard compared to the catheter body such that the tip cannot be bent or deformed. With reference to FIG. 25, in this embodiment, the distal region 166 may be at least as rigid or more rigid than the ultrasound radiating member region 164. In one embodiment, the length of the distal region 166′ is less than about 0.25 inches and in another embodiment less than about 0.1 inches.

Other aspects of the ultrasound catheter distal tip design can be manipulated to reduce the length of the distal rigid section, and therefore to enhance the maneuverability of the ultrasound catheter. For example, the ability of the ultrasound catheter to reliably track the guidewire can be improved by reducing the wicking of adhesive 132 (see FIG. 2A) in the region of the proximal element joint. This can be accomplished by using less adhesive 132 in the proximal element joint, and/or by modifying the bonding methods and techniques at the proximal element joint, as described herein. The strength of the proximal element joint can be maintained with less adhesive by increasing the “overlap” between the sleeve 130 and the outer sheath 108.

Benchmarking.

Ultrasound catheters manufactured according to the various embodiments provided herein and, in particular, the improvements described with reference to FIGS. 26-29 have advantageous physical properties that facilitate delivery of the catheter to a treatment site located within a patient's distal vasculature. The mechanical properties of these catheters, such as stiffness, guidewire movement, and other properties, can be tested using standard testing equipment, such as tensile testers, force gauges, and Tinius Olsen stiffness testers. Catheter designs can be evaluated in a water bath at approximately 37° C. to simulate conditions encountered within a patient's vasculature.

For example, the stiffness of the catheter as a function of axial catheter position can be determined using an Instron® tensile strength testing machine. In one exemplary embodiment, the stiffness of the ultrasound catheter is less than about 0.05 pounds in a region within 20 cm from the distal catheter tip. In another exemplary embodiment, the stiffness of the ultrasound catheter is less than about 0.15 pounds in a region within 20 cm from the distal tip. In another exemplary embodiment, the stiffness of the ultrasound catheter is less than about 0.10 pounds in a region within 30 cm from the distal tip. In another exemplary embodiment, the stiffness of the ultrasound catheter is less than about 0.20 pounds in a region within 30 cm from the distal tip.

Guidewire movement, which can be hindered by kinking or distortion (also referred to as “ovalization”) of the catheter body, can be determined by observing guidewire movement through loops and/or curves of varying diameter. For example, in one test, a standard 0.014 inch guidewire is passed through a catheter bent into one or more 360° loops having diameters of between about 6 mm and about 12 mm. Such loops are representative of the tortuosity encountered in accessing a typical treatment site, such as the middle cerebral artery. In another test, the catheter is bent into a series of S-curves. As the guidewire is pushed and pulled through the loop/curve, any drag, bumps or wire flexure is observed, which may indicate a kink in the catheter, ovalization of the catheter, binding of the guidewire, or some other deleterious condition.

The ability of the ultrasound catheter to track the guidewire at a difficult region of the patient's vasculature, such as at a small radius bend or at a bifurcation, can also be evaluated. Generally, a greater force is required to navigate the catheter around a small-radius curved path than a large-radius curved path; and generally a greater force is required to navigate the ultrasound catheter around a 180° curve than a curve less than 180°. For example, in one embodiment, less than approximately 10 grams are required to pull an ultrasound catheter over a standard 0.014 inch guidewire around a 7 mm diameter curve. In another embodiment, less than approximately 8 grams are required to pull an ultrasound catheter over a standard 0.014 inch guidewire around a 7 mm diameter curve.

These improvements allow the ultrasound catheter disclosed herein to consistently and safely reach the distal regions of a patient's neurovascular system, including, but not limited to, the main and subsequent branches of the middle cerebral artery. This represents a significant advancement in ultrasound catheters.

In addition to the flexibility improvements described above, it is also advantageous that the flexibility characteristics described above are achieved with an ultrasound catheter that provides enough room for a transducer element of sufficient size to deliver a therapeutically sufficient dose of ultrasound energy. Accordingly, in one embodiment, the ultrasound catheter has a utility lumen inner diameter of less than about 0.018 inches, and in another embodiment, less than about 0.017 inches and yet still capable of being able to receive a standard 0.014 inch guidewire. In such an embodiment, the outer diameter of the region of the catheter comprising the ultrasound radiating member, has a diameter of greater than about 2.0 French, and in another embodiment, a diameter that is greater than about 2.8 French, and in another embodiment greater than about 3.3 French. This arrangement advantageously allows a sufficiently large transducer element to advanced through small blood vessels, such as the main and subsequent branches of the middle cerebral artery.

Conclusion.

While the foregoing detailed description has set forth several exemplary embodiments of the apparatus and methods of the present invention, it should be understood that the above description is illustrative only and is not limiting of the disclosed invention. It will be appreciated that the specific dimensions and configurations disclosed can differ from those described above, and that the methods described can be used within any biological conduit within the body.

1. An ultrasound catheter configured to be advanced into a patient's neurovascular system, comprising:
an elongate outer sheath defining a central lumen that extends longitudinally from an outer sheath proximal region to an outer sheath distal region;
an elongate hollow inner core positioned in the central lumen, the inner core defining a utility lumen configured to receive a guidewire, the inner core having a distal region that terminates at a point that is proximal to the outer sheath distal region, the inner core comprising a reinforcing member extending along at least a portion of the inner core, the reinforcing member being configured to reduce ovalization of the inner core as the catheter is bent;
a tubular inner support member coupled to the inner core distal region;
a tubular outer support member coupled to the outer sheath distal region; and
a ultrasound radiating member having an inner passage, wherein the ultrasound radiating member is positioned generally the between the inner and outer support members, such that the inner support member passes through the hollow inner core and the outer support member is positioned over an outer surface of the ultrasound radiating member.

2. The ultrasound catheter of claim 1, wherein the inner core comprises an inner member and outer member and at least a portion of the reinforcing member is positioned in a space between the inner and outer members.

3. The ultrasound catheter of claim 1, further comprising a distal tip coupled to at least one of the inner and outer tubular support members.

4. The ultrasound catheter of claim 3, wherein the distal tip extends less than about 0.5 inches beyond the ultrasound radiating member.

5. The ultrasound catheter of claim 3, wherein the distal tip extends less than 1 mm past the ultrasound radiating member.

6. The ultrasound catheter of claim 1, further comprising an adhesive bond configured to couple the outer sheath distal region to the tubular outer support member.

7. The ultrasound catheter of claim 6, wherein the outer sheath distal region includes a reduced diameter portion, the outer support member and the adhesive bond extending at least partially over the reduced diameter portion.

8. The ultrasound catheter of claim 7, wherein the tubular outer support member is positioned over the reduced diameter portion of the outer sheath distal region in a slip-fit arrangement, such that the tubular outer support member has an outer diameter that is less than or equal to an outer diameter of the outer sheath proximal portion.

9. The ultrasound catheter of claim 1, wherein the tubular inner support member comprises a polyimide tube.

10. The ultrasound catheter of claim 1, wherein the reinforcing member is wrapped around a portion of the elongate inner core in a helical pattern.

11. The ultrasound catheter of claim 10, wherein the reinforcing member comprises a stainless steel wire with a flattened profile.

12. An neurovascular catheter, comprising:
a tubular body having a proximal a distal end; the tubular body comprising:
an outer tubular component having a proximal region, a distal region end and a lumen extending therethrough,
an inner tubular component positioned within the lumen of the outer tubular component and extending from the proximal region to distal region of the outer tubular component, the inner tubular component forming, at least in part a utility lumen, that extends from proximal end of the tubular body to the distal end of the tubular body, the inner tubular body being formed at least in part from a composite tube comprising an inner member, an outer member and a reinforcing coil which is positioned between the outer member and the inner member;
at least one ultrasound radiating member positioned generally between the outer tubular component and the inner tubular component at the distal end of the tubular body.

13. The neurovascular catheter of claim 13, wherein the inner member comprises Teflon.

14. The neurovascular catheter of claim 13, wherein the outer member comprises Pebax.

15. The neurovascular catheter of claim 13, wherein the reinforcing coil comprises a stainless steel.

16. The neurovascular catheter of claim 13, wherein the ultrasound radiating member comprises a cylindrical ultrasound radiating member with a through bore through which at least a portion of the inner tubular component extends.

17. The neurovascular catheter of claim 13, further comprising a temperature sensor positioned adjacent to the ultrasound radiating member.

18. The neurovascular catheter of claim 13, wherein the composite tube is configured such that the reinforcing coil extends over at least at least 50% of the length of the catheter.

19. The neurovascular catheter of claim 13, wherein the composite tube is configured such that the reinforcing coil extends over at least 75% of the length of the catheter.

20. The neurovascular catheter of claim 13, wherein the composite tube is configured such that the reinforcing coil extends over substantially the entire length of the catheter.

21. A catheter having a distal end and a proximal end comprising;
an elongate outer sheath with an exterior surface, wherein the distal end portion of said outer sheath has an outer diameter of less than about 5 French for advancement through a small blood vessel, said outer sheath defining a central lumen extending longitudinally therethrough;
an elongate inner core extending through said central lumen of said outer sheath and terminating at an exit port located at the distal end of the catheter; said inner core defining a utility lumen adapted to receive a guidewire;
an ultrasound member positioned at the distal end of the catheter body generally between the outer sheath and the inner core; and
a guidewire configured to be slideably received within the utility lumen for advancement of the catheter to a treatment site, the guidewire having a diameter that is less than or equal to about 0.017 inches;
wherein the catheter is configured such that the catheter an subjected to a 180 degree bend having a radius of less than about 10 mm while still permitting the catheter to slide over the guidewire.

22. The catheter of claim 21, wherein the catheter is configured such that the catheter an subjected to a 180 degree bend having a radius of less than about 8 mm while still permitting the catheter to slide over the guidewire.

23. The catheter of claim 22, the catheter is configured such that the catheter an subjected to a 180 degree bend having a radius of less than about 6 mm while still permitting the catheter to slide over the guidewire.

24. The catheter of claim 23, wherein the distal end portion of said outer sheath has an outer diameter of greater than about 2.3 French.

25. An neurovascular catheter, comprising:
a tubular body having a proximal a distal end; the tubular body comprising:
an outer tubular component having a proximal region, a distal region end and a lumen extending therethrough,
an inner tubular component positioned within the lumen of the outer tubular component and extending from the proximal region to distal region of the outer tubular component, the inner tubular component forming, at least in part a utility lumen, that extends from proximal end of the tubular body to the distal end of the tubular body;
at least one ultrasound transducer positioned generally between the outer tubular component and the inner tubular component at the distal end of the tubular body; and
at least one electrical wire electrically coupled to the ultrasound transducer and extending between the outer tubular component and the inner tubular component, at least one electrical wire extending from the proximal region to distal region of the outer tubular component;
wherein a portion of the electrical wire extending between the inner and outer tubular components has an extended length that is at least 0.02% longer than the extended length of the corresponding portion of the inner and outer tubular components.

26. The neurovascular catheter of claim 25, wherein the portion of the electrical wire extending between the inner and outer tubular components has extended length that is greater than about 0.5% longer than the extended length of the corresponding portion of the inner and outer tubular components.

27. The neurovascular catheter of claim 25, wherein the portion of the electrical wire extending between the inner and outer tubular components is wrapped around the inner tubular component in a helical pattern.

28. The neurovascular catheter of claim 25, further comprising a temperature sensor positioned generally between the outer tubular component and the inner tubular component at the distal end of the tubular body, the temperature sensor operatively connected to a control system through a control wire having a extending between the inner and outer tubular components that has an extended length that is at least 0.02% longer than the extended length of the corresponding portion of the inner and outer tubular components.

29. A neurovascular catheter configured be advanced to a vascular occlusion positioned in the patient's neurovascular system;
an elongate body having a proximal region, a distal region opposite the proximal region and a distal tip region; the body becoming generally more flexible from the proximal region to the distal region, the distal region having an outer diameter less than about 5 French; and
an energy delivery device within the distal region of the and configured to delivery energy to the treatment site;
wherein the distal tip region includes a substantially rigid portion that cannot bend during normal use conditions, the substantially rigid portion having a length that less than about 6 mm.

30. The neurovascular catheter of claim 29, wherein the length is of the substantially rigid portion is greater than about 3 mm.

31. The neurovascular catheter of claim 29, wherein the length is of the substantially rigid portion is less than about 5 mm.

32. The neurovascular catheter of claim 29 wherein the length is of the substantially rigid portion is greater than about 4 mm

33. A method of manufacturing an ultrasound catheter, the method comprising:
providing an elongate outer sheath that defines a central lumen extending longitudinally from an outer sheath proximal region to an outer sheath distal region;
providing a plurality of elongate electrical conductors within the central lumen;
positioning an elongate inner core in the central lumen, such that the plurality of electrical conductors are positioned between the inner core and the outer sheath;
coupling a tubular inner support member to a distal region of the elongate inner core;
mounting an ultrasound radiating member to the inner support member, wherein the ultrasound radiating member includes a hollow inner core through which the inner support member is positioned; and
coupling a tubular outer support member to a distal region of the elongate outer sheath, wherein the tubular outer support member is positioned over an outer surface of the ultrasound radiating member.

34. The method of claim 33, further comprising electrically coupling the elongate electrical conductors to the ultrasound radiating member.

35. The method of claim 33, further comprising positioning a temperature sensor adjacent to the ultrasound radiating member.

36. The method of claim 33, wherein the tubular outer support member is coupled to the distal region of the elongate outer sheath using an epoxy.

37. The method of claim 33, wherein the tubular inner support member is coupled to the distal region of the elongate inner using a slip-fit configuration.

38. The method of claim 33, further comprising coupling a distal tip to one or more of the inner and outer tubular support members, wherein the distal tip is positioned distal to the ultrasound radiating member.
